(19) United States
(12) Patent Application Publication
(10) Pub. No.: US 2026/0124053 A1
(43) Pub. Date: May 7, 2026

(54) RELEASABLE GUIDE MEMBERS FOR PRE-CANNULATING BRANCHED STENT GRAFT DELIVERY

(71) Applicant: W. L. Gore & Associates, Inc., Newark, DE (US)

(72) Inventors: Michael R. Broyles, Flagstaff, AZ (US); Daniel S. Cole, Flagstaff, AZ (US); Michael J. Shepard, Flagstaff, AZ (US)

(21) Appl. No.: 19/377,908

(22) Filed: Nov. 3, 2025

Related U.S. Application Data
(60) Provisional application No. 63/716,035, filed on Nov. 4, 2024.
(60) Provisional application No. 63/837,314, filed on Jul. 2, 2025.

(51) Int. Cl. A61F 2/954; A61F 2/95

(52) U.S. Cl. CPC A61F 2/954; A61F 2002/9511

(57) ABSTRACT

A delivery system for an implantable device includes an elongate member and a main body coupled to the elongate member. The main body has a main body wall defining a main body lumen and a side branch portal through the main body wall. A guide member extends along at least a portion of the elongate member, into the main body lumen, and through the side branch portal. A guide member retainer assembly includes a retainer coupled to the guide member and defining a retainer eyelet. A release wire extends through a portion of the main body wall and the retainer eyelet to engage the retainer eyelet, restricting the guide member from being retracted along the elongate member by the engagement between the release wire and the retainer eyelet.

### CROSS-REFERENCE TO RELATED APPLICATION

This application claims the benefit of Provisional Application No. 63/837,314, filed Jul. 2, 2025, and also claims the benefit of Provisional Application No. 63/716,035, filed Nov. 4, 2024, which are incorporated herein by reference in their entireties for all purposes.

### FIELD

The present disclosure relates to delivery systems for implantable medical devices, and more particularly to a delivery system for deploying a branched stent graft with independently releasable guide members for pre-cannulating side branch portals.

### BACKGROUND

A variety of branched, anatomical passages may benefit from treatment in the form of an implanted, endoluminal device. One such passage is a vascular passage, such as an artery, with an aneurysm. Aortic disease and trauma such as aneurysms and dissections present a significant risk to a patient. That risk is increased based on the patient's condition. Such conditions or factors can include the patient's age and preexisting and/or related conditions such as cardiopulmonary bypass, cardiac arrest, circulatory arrest. These and other factors may limit the patient's ability to withstand and recover from surgery to repair the aortic disease. This same issue exists in other diseased and damaged tissues in the patients.

With respect to aneurysms, in order to prevent rupturing of an aneurysm, a stent graft may be introduced into a blood vessel percutaneously and deployed to span the aneurysmal sac. Stent grafts include a graft fabric secured to a cylindrical scaffolding or framework of one or more stents. The stent(s) provide rigidity and structure to hold the graft open in a tubular configuration as well as the outward radial force needed to create a seal between the graft and a healthy portion of the vessel wall and provide migration resistance. Blood flowing through the vessel can be channeled through the luminal surface of the stent graft to reduce, if not eliminate, the stress on the vessel wall at the location of the aneurysmal sac. Stent grafts may reduce the risk of rupture of the blood vessel wall at the aneurysmal site and allow blood to flow through the vessel without interruption.

Various endovascular repair procedures such as the exclusion of an aneurysm require a stent graft to be implanted adjacent to a vascular bifurcation. Often the aneurysm extends into the bifurcation requiring the stent graft to be placed into the bifurcation. A bifurcated stent graft is therefore required in these cases. Modular stent grafts, having a separate main body and branch component are often preferred in these procedures due to the ease and accuracy of deployment. See U.S. Patent Application No. 2008/0114446 to Hartley et al. for an example of a modular stent graft having separate main body and branch stent components. In the Hartley et al. publication the main body stent has a fenestration in the side wall that is tailored to engage and secure the side branch stent.

However, delivery and deployment of side branches through a main body can be difficult. For example, delivering side branch devices through the appropriate openings in the main body and into the appropriate side branch vessel can be difficult in endovascular procedures.

Additionally, management of wires for delivering branch stent components to the target site can be difficult as the wires can become entangled during delivery. Additionally, the wires can be obstructive of procedures and movements during delivery and deployment of the devices.

### SUMMARY

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

According to an example (“Example 1”), a delivery system for an implantable device includes an elongate member, a main body coupled to the elongate member, a guide member, and a guide member retainer assembly. The main body includes a main body wall defining a main body lumen and a side branch portal through the main body wall. The guide member extends along at least a portion of the elongate member, into the main body lumen, and through the side branch portal. The guide member retainer assembly includes a retainer coupled to the guide member and defining a retainer eyelet, and a release wire extending through a portion of the main body wall and the retainer eyelet to engage the retainer eyelet such that the guide member is restricted from being retracted along the elongate member by the engagement between the release wire with the retainer eyelet.

According to another example (“Example 2”), further to Example 1, the retainer is defined at a proximal end of the guide member.

According to another example (“Example 3”), further to Example 2, the retainer eyelet is defined through a body of the proximal end of the guide member.

According to another example (“Example 4”), further to Example 1, the guide member retainer assembly includes a plurality of retainers arranged in a nested configuration.

According to another example (“Example 5”), further to Example 4, the plurality of retainers are positioned at different longitudinal positions along the release wire.

According to another example (“Example 6”), further to Example 5, the plurality of retainers are positioned relative to each other to distribute multiple guide members in a manner that corresponds to longitudinal and angular positioning of multiple side branch portals in the main body.

According to another example (“Example 7”), further to Example 1, the retainer has a thinner profile than a remainder of the guide member.

According to an example (“Example 8”), a delivery system for an implantable device includes an elongate member, a main body positioned about the elongate member, a guide member, and a guide member retainer assembly. The main body includes a frame. The guide member extends along at least a portion of the elongate member. The guide member retainer assembly releasably couples the guide member to the frame and includes a retainer coupled to the guide member and defining a side wall opening, and a release wire extending through the guide member and the side wall opening such that the frame is positioned between the guide member and the release wire at the side wall opening to releasably couple the guide member to the frame.

According to another example (“Example 9”), further to Example 8, the retainer comprises a proximal hypotube coupled to a proximal end of the guide member.

According to another example (“Example 10”), further to Example 9, the retainer further comprises a second proximal hypotube positioned within the proximal hypotube, the proximal hypotube and the second proximal hypotube configured to translate relative to each other to release the frame.

According to another example (“Example 11”), further to Example 9, the retainer further comprises a first side wall opening and a second side wall opening positioned along a length of the proximal hypotube, wherein a frame retainer groove is formed between the first and second side wall openings.

According to another example (“Example 12”), further to Example 11, the release wire extends through the guide member, out the first side wall opening, and back into the second side wall opening to secure the frame in the frame retainer groove.

According to another example (“Example 13”), further to Example 12, the retainer further comprises a loop coupled to the proximal hypotube, the loop including a retainer ball, wherein the proximal hypotube includes a sidewall aperture configured to receive the retainer ball, and wherein the release wire engages the retainer ball to secure the frame until the release wire is withdrawn.

According to another example (“Example 14”), further to Example 9, the proximal hypotube further comprises retainer protrusions extending partially into the side wall opening, the retainer protrusions configured to flex to allow passage of the release wire through the side wall opening when a threshold force is applied.

According to an example (“Example 15”), a delivery system for an implantable device includes an elongate member, a main body positioned about the elongate member, a guide member, and a guide member retainer assembly. The main body includes a frame and defines a side branch member. The guide member is positioned through the side branch member in a pre-cannulated configuration. The guide member retainer assembly releasably couples the guide member to the frame and includes a proximal hypotube including a slot and a retention tab, and a release wire extending through the proximal hypotube, the release wire including a portion protruding from the slot and a groove engaged by the retention tab.

According to another example (“Example 16”), further to Example 15, the delivery system further comprises a disengagement tube configured to disengage the retention tab from the groove when advanced proximally, allowing the release wire to be withdrawn to release the frame.

According to another example (“Example 17”), further to Example 16, the retention tab bends inwardly to define a ramp surface.

According to another example (“Example 18”), further to Example 17, the disengagement tube is configured to translate along the ramp surface and deflect the retention tab away from the groove to release the release wire from its longitudinally constrained position relative to the proximal hypotube.

According to another example (“Example 19”), further to Example 15, the release wire includes a proximal end that is constrained by the proximal hypotube when the retention tab is engaged with the groove, and wherein withdrawal of the release wire results in the proximal end being freed from the proximal hypotube.

According to another example (“Example 20”), further to Example 15, the curved portion is configured to engage with the frame when the retention tab is engaged with the groove, and wherein advancement of the disengagement tube causes progressive disengagement of the retention tab from the groove to allow withdrawal of the release wire and release of the frame from the guide member retainer assembly.

According to an example (“Example 21”) A delivery system includes an elongate member; an implantable device including a main body defining a main lumen and a side branch portal, the implantable device arranged about at least a portion of the elongate member; a guide member extending along at least a portion of the elongate member and extending through the side branch portal, the guide member being configured to guide movement of a branch body; a lockwire; and a guide member retainer assembly, wherein the lockwire is configured to be releasably engaged with the guide member retainer assembly such that the guide member is restricted from being retracted along the elongate member and through the side branch portal when the lockwire is in engagement with the guide member retainer assembly.

According to another example (“Example 22”) further to Example 21, a coupling portion of the guide member and the guide member retainer assembly includes an end cap positioned at a proximal end of the elongate member.

According to another example (“Example 23”) further to Example 22, the guide member retainer assembly includes an engagement portion that is releasably coupled to the end cap.

According to another example (“Example 24”) further to Examples 21-23, the guide member includes a guide member first end portion that is opposite a guide member second end portion, wherein the guide member retainer assembly includes an engagement portion that is a loop formed at the guide member second end portion, and wherein the lockwire is received through the loop.

According to another example (“Example 25”) further to Examples 21-24, the guide member retainer assembly defines an orifice in the end cap through which the guide member is received to be secured at a securable portion of the guide member.

According to another example (“Example 26”) further to Example 25, the orifice is formed as a slot that includes a free end through which the guide member is received to be secured at a guide member securable portion of the guide member.

According to another example (“Example 27”) further to Example 26, the guide member securable portion is an embossment that is sized to inhibit the guide member from being released through the orifice.

According to another example (“Example 28”) further to Examples 25-27, the slot is formed through at least a portion of a thickness of the end cap.

According to another example (“Example 29”) further to Examples 22-28, a lockwire is received through a lockwire orifice formed in the end cap.

According to another example (“Example 30”) further to Example 29, the lockwire and end cap further include a movable collar movably coupled to the end cap, and wherein the lockwire orifice is formed in the movable collar to secure the movable collar to the end cap.

According to another example (“Example 31”) further to Example 30, the movable collar is allowed to move relative to the elongate member when the lockwire is withdrawn through the lockwire orifice.

According to another example (“Example 32”) further to Example 31, the movable collar is in a threaded engagement with the end cap such that withdrawal of the lockwire allows the movable collar to unthread from the elongate member.

According to another example (“Example 33”) further to Example 32, the movable collar is unthreaded from the end cap by tensioning the guide member.

According to another example (“Example 34”) further to Examples 21-33, the guide member is one of a plurality of guidewires and the branch body is one of a plurality of branch bodies, a number of guidewires in the plurality of guidewires corresponding to a number of branch bodies in the plurality of branch bodies, and wherein each of the guidewires in the plurality of guidewires is engaged by a lockwire.

According to another example (“Example 35”) further to Example 34, the side branch portal includes an opening that faces in a distal direction relative to the main body.

According to an Example (“Example 36”), a delivery system for a multibranch endoprosthesis includes an elongate member including an elongate member body with an elongate member first end portion and an elongate member second end portion that opposes the elongate member first end portion, the elongate member body including an elongate member wall defining an elongate member lumen; a plurality of guide members extending along at least a portion of the elongate member body; and a guide member retainer assembly releasably engaging the plurality of guide members, the guide member retainer assembly including a movable collar and an end cap, the movable collar operable to restrict movement of the plurality of guide members when in an engaged configuration.

According to another example (“Example 37”) further to Example 36, the end cap defines slots through which the plurality of guide members extend and free ends positioned proximate the movable collar when the movable collar is in the engaged configuration.

According to another example (“Example 38”) further to Example 37, the guide member retainer assembly further includes a lockwire removably engaged with the movable collar and end cap, wherein movable collar is operable to move away from the free ends when the lockwire is removed from the movable collar and end cap.

According to an Example (“Example 39”), a method of deploying a multibranch endoprosthesis at a target site including a main lumen and at least one branch lumen, the method including advancing a main guidewire to a target site; advancing an elongate member including an implantable device having a side branch portal, the side branch portal being precannulated with a guide member; advancing at least one of a catheter and a side branch along the guide member; actuating a lockwire of a guide member retainer assembly to release a movable collar from an engaged configuration to a disengaged configuration, the movable collar configured to restrain the guide member from being retracted when the movable collar is in the engaged configuration; and tensioning the guide member to cause movement of the movable collar and release the guide member from the guide member retainer assembly.

According to another Example (“Example 40”), further to Example 39, tensioning the guide member includes causing the movable collar to simultaneously rotate and move longitudinally relative to the elongate member.

The foregoing general description of the illustrative embodiments and the following detailed description thereof are merely exemplary aspects of the teachings of this disclosure and are not restrictive.

### BRIEF DESCRIPTION OF FIGURES

Non-limiting and non-exhaustive examples are described with reference to the following figures.

FIG. 1 illustrates a side view of a delivery system for deploying an implantable device, according to aspects of the present disclosure.

FIG. 2 depicts a section view of a portion of human anatomy showing the aorta and its major branches, according to an embodiment.

FIG. 3 illustrates a side view of an implantable device positioned within an aorta, according to aspects of the present disclosure.

FIG. 4 depicts a side view of a filtration system deployed within an anatomical vessel structure, according to an embodiment.

FIG. 5 illustrates a portion of a delivery system with multiple guide members, according to aspects of the present disclosure.

FIG. 6 depicts a portion of a medical device with a sheath for each guide member, according to an embodiment.

FIG. 7 illustrates a portion of a medical device showing an articulatable guide member assembly, according to aspects of the present disclosure.

FIGS. 8A-9B depict orthogonal views of a guide member retainer assembly, according to various embodiments.

FIG. 10 illustrates a view of a guide member retainer assembly showing interaction with a stent graft, according to an aspect of the present disclosure.

FIG. 11 depicts a side view of a delivery system for deploying a medical device, according to an embodiment.

FIGS. 12A-12B illustrate orthogonal views of a guide member retainer assembly, according to aspects of the present disclosure.

FIGS. 13A-13C depict orthogonal views of a guide member retainer assembly engaging with a frame, according to various embodiments.

FIGS. 14A-14C illustrate various views of a guide member retainer assembly for securing a guide member, according to aspects of the present disclosure.

FIGS. 15A-15D depict orthogonal views of a guide member retainer assembly showing different configurations, according to various embodiments.

FIG. 16 illustrates a section view of a guide member retainer assembly engaged with a frame, according to an aspect of the present disclosure.

FIGS. 17A-17B depict orthogonal views of a guide member retainer assembly with a proximal hypotube, according to embodiments.

FIGS. 18A-18B illustrate orthogonal and section views of a guide member retainer assembly with a frame retainer groove, according to aspects of the present disclosure.

FIGS. 19A-19B depict orthogonal and section views of a guide member retainer assembly with a loop, according to embodiments.

FIGS. 20A-20B illustrate orthogonal and section views of a guide member retainer assembly with a retainer ball, according to aspects of the present disclosure.

FIGS. 21A-21C depict various views of a guide member retainer assembly with a second proximal hypotube, according to embodiments.

FIGS. 22A-22B illustrate orthogonal and section views of a guide member retainer assembly with a slot, according to aspects of the present disclosure.

FIGS. 23A-23C depict orthogonal and section views of a guide member retainer assembly with a splaying feature, according to embodiments.

FIGS. 24A-24B illustrate orthogonal and section views of a guide member retainer assembly with a second proximal hypotube having a retainer, according to an aspect of the present disclosure.

FIG. 25 depicts an orthogonal view of a guide member retainer assembly with a pin coupled to a release wire, according to embodiments.

FIGS. 26A-27 illustrate orthogonal views of a guide member retainer assembly with a second proximal hypotube and a pin, according to aspects of the present disclosure.

FIGS. 28A-28F depict various views of a guide member retainer assembly showing a release sequence, according to embodiments.

FIGS. 29A-29B illustrate photographs of the guide member retainer assembly of FIGS. 28A-28F coupled to a frame, according to aspects of the present disclosure.

FIGS. 30A-30D depict several views of a guide member retainer assembly in more detail, according to embodiments.

FIGS. 31A-31B illustrate orthogonal views of a guide member retainer assembly with a disengagement tube, according to aspects of the present disclosure.

FIGS. 32A-32D depict various views of a guide member retainer assembly with retainer protrusions, according to embodiments.

FIGS. 33A-33B illustrate side views of a guide member retainer assembly with a removal member, according to aspects of the present disclosure.

FIGS. 34A-34B depict side views of a guide member retainer assembly with a coupling portion, according to embodiments.

FIGS. 35-36E are views of an implantable device with guide members secured to a tip/olive and extending though a side branch portal access.

FIGS. 37-39 are views of steps in a multistage endoprosthesis deployment process of a multibranch endoprosthesis showing how to release guide members from a lockwire assembly.

### DETAILED DESCRIPTION

This disclosure is not meant to be read in a restrictive manner. For example, the terminology used in the application should be read broadly in the context of the meaning those in the field would attribute such terminology.

Persons skilled in the art will readily appreciate that various aspects of the present disclosure can be realized by any number of methods and apparatus configured to perform the intended functions. Stated differently, other methods and apparatus can be incorporated herein to perform the intended functions. It should also be noted that the accompanying drawing figures referred to herein are not necessarily drawn to scale, but may be exaggerated to illustrate various aspects of the present disclosure, and in that regard, the drawing figures should not be construed as limiting.

Certain relative terminology is used to indicate the relative position of components and features. For example, words such as “top”, “bottom”, “upper,” “lower,” “left,” “right,” “horizontal,” “vertical,” “upward,” and “downward” are used in a relational sense (e.g., how components or features are positioned relative to one another) and not in an absolute sense unless context dictates otherwise. Similarly, throughout this disclosure, where a process or method is shown or described, the method may be performed in any order or simultaneously, unless it is clear from the context that the method depends on certain actions being performed first.

With respect to terminology of inexactitude, the terms “about” and “approximately” may be used, in certain instances, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error, differences in measurement and/or manufacturing equipment calibration, human error in reading and/or setting measurements, minor adjustments made to optimize performance and/or structural parameters in view of differences in measurements associated with other components, particular implementation scenarios, imprecise adjustment and/or manipulation of objects by a person or machine, and/or the like, for example.

As used herein, “couple” means join, connect, attach, adhere, affix, or bond, whether directly or indirectly, and whether permanently or temporarily.

The term “film” as used herein generically refers to one or more of the membrane, composite material, or laminate.

The term “biocompatible material” as used herein generically refers to any material with biocompatible characteristics including synthetic materials, such as, but not limited to, a biocompatible polymer, or a biological material, such as, but not limited to, bovine pericardium. Biocompatible material may comprise a first film and a second film as described herein for various embodiments.

For reference, the terms “circumference” and “diameter” are not meant to require a circular cross-section (although are inclusive of a circular cross-section), and are instead to be understood broadly to reference an outer surface or dimension and the dimension between opposing sides of the outer surface, respectively.

Although the embodiments herein may be described in connection with various principles and beliefs, the described embodiments should not be bound by theory. For example, embodiments are described herein in connection with vascular stent grafts, and more specifically branched stent grafts. However, embodiments within the scope of this disclosure can be applied toward any endoprostheses of similar structure and/or function. Furthermore, embodiments within the scope of this disclosure can be applied in non-vascular applications.

### Description of Various Embodiments

Persons skilled in the art will readily appreciate that various aspects of the present disclosure can be realized by any number of methods and apparatuses configured to perform the intended functions. It should also be noted that the accompanying drawing figures referred to herein are not necessarily drawn to scale, but may be exaggerated to illustrate various aspects of the present disclosure, and in that regard, the drawing figures should not be construed as limiting.

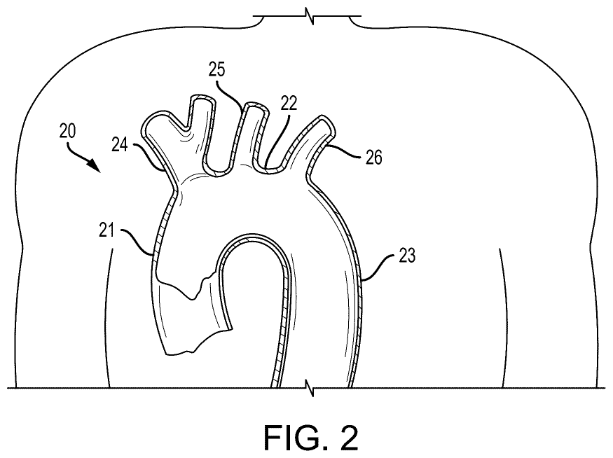

Devices, systems, and methods of endoluminally delivering an endoprosthesis including a branchable expandable implant in accordance with various embodiments are disclosed herein for treating disease of human vasculature. Although the description below and figures are illustrated in the context of treating the aorta 20, including the ascending aorta 21, aortic arch 22, and descending aorta 23, and branches therefrom, including the brachiocephalic artery 24, the left common carotid artery 25, and the left subclavian artery 26 (see FIG. 2), it should be appreciated that the present disclosure can be applied to treatment of other portions of the vasculature or, including, for example, any disease where a larger vessel and one or more branch vessels are to be treated.

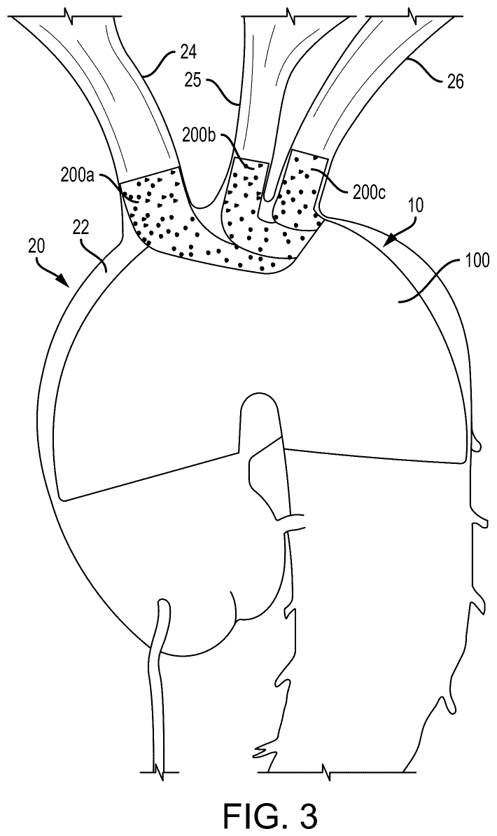

Referring to FIG. 3, an implantable device 10 can be delivered and deployed in the aorta 20, the implantable device 10 including a main body 100 and branch bodies 200. The main body 100 is deployed in the aortic arch 22 and the branch bodies 200 can be deployed in branching arteries (e.g., a first branch body 200a in the brachiocephalic artery 24, a second branch body 200b in the left common carotid artery 25, and a third branch body 200c in the left subclavian artery 26).

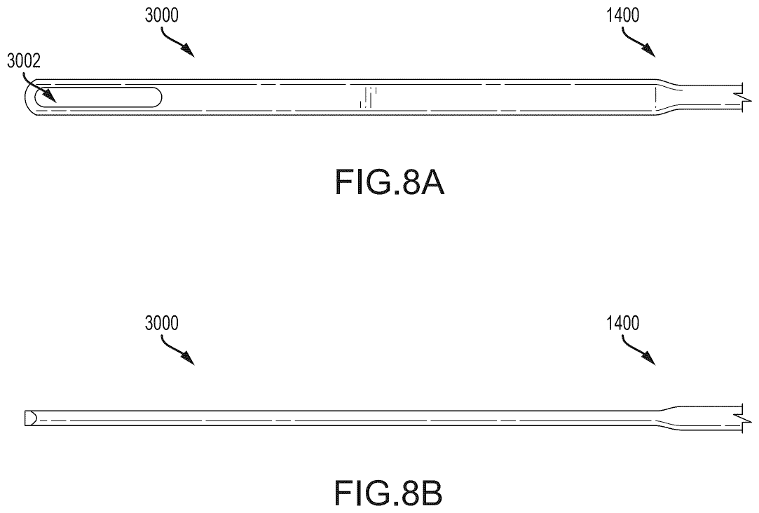

Although various configurations of the implantable device 10 are contemplated with respect to the delivery systems and methods described herein, several discrete examples of an implantable device 10 are provided in detail in order to provide reference for the various components and steps of the delivery system and method of delivery and deployment. As seen in FIG. 8, the main body 100 includes branch bodies 200 extending through a portion of the side wall. More specifically, the branch bodies extend through side branch portals defined through the side wall, the side branch portals being configured to receive the branch bodies 200 therethrough.

The present disclosure relates to various portions of the delivery system for effectively endovascularly delivering branch bodies 200 to branched vasculature. More specifically, branch bodies 200 are delivered to bridge the vasculature from a main body 100 positioned at a target position in the main vessel to the branching vessel. The devices described herein facilitate simplified and straightforward delivery of branch bodies 200 through branches or fenestrations in the main body 100 and into branching vessels. The devices provide the ability to cannulate branching vessels in preparation for delivery of devices into a branching vessel. The devices facilitate simple withdrawal of procedural aids during the procedure in preparation for deployment of expandable devices.

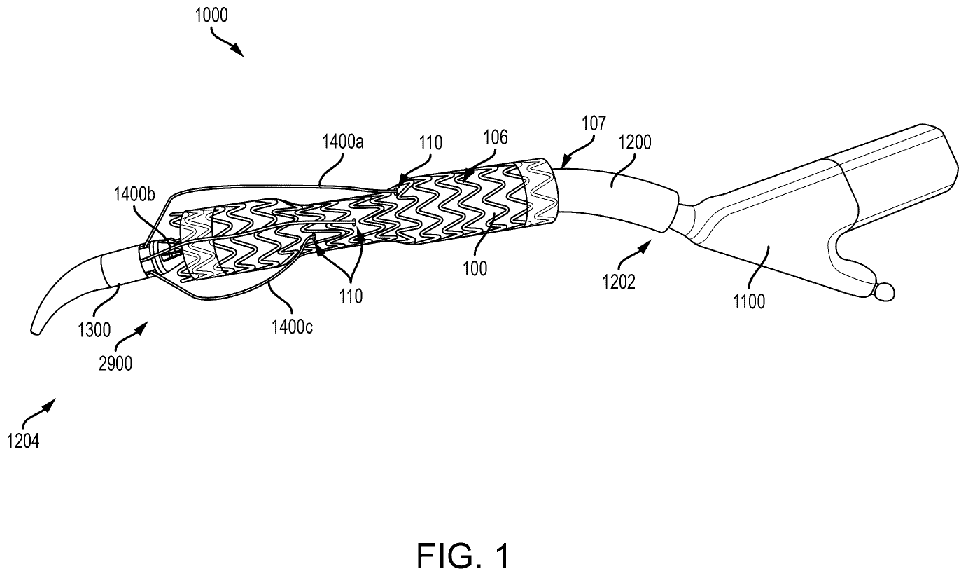

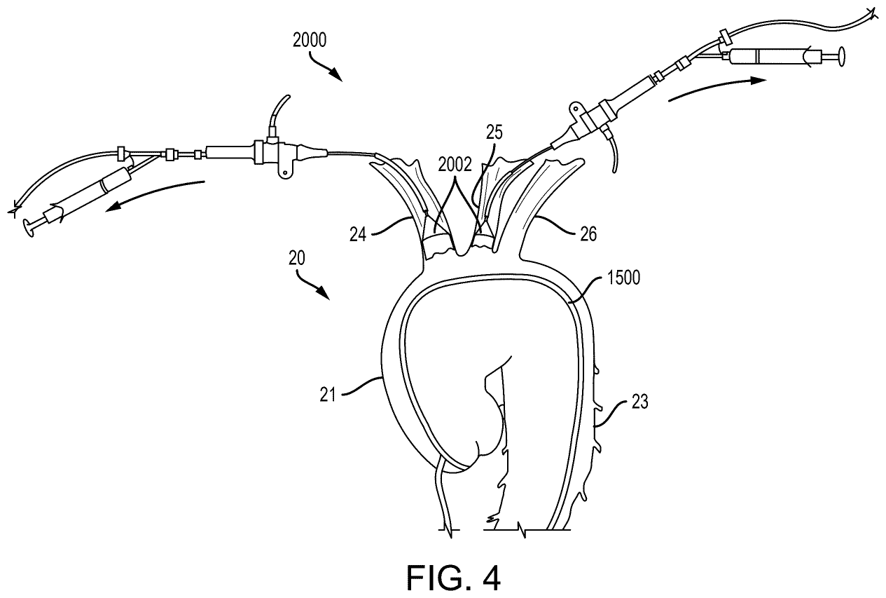

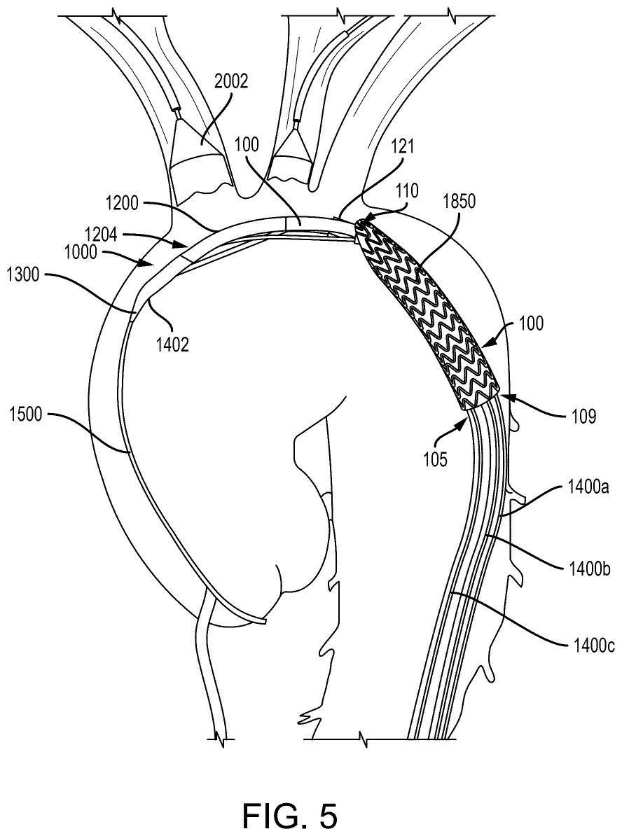

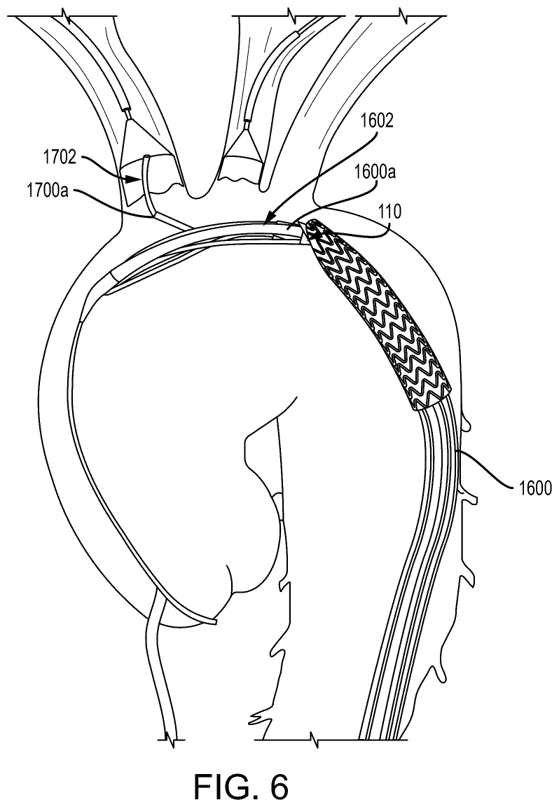

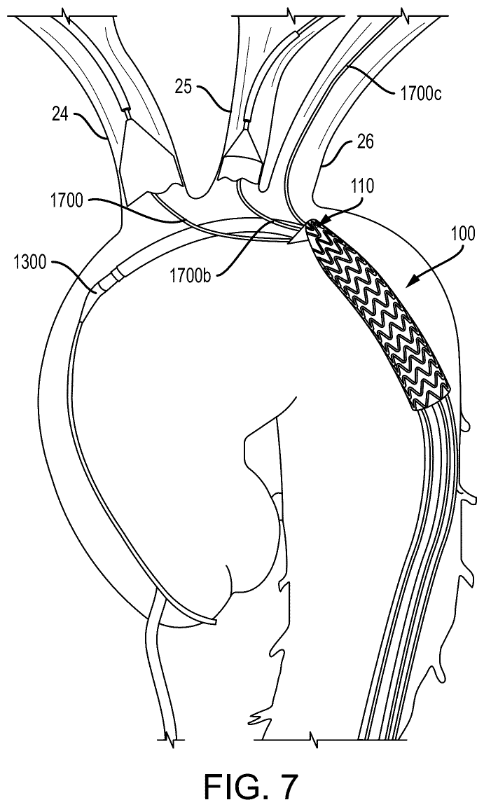

In order to better understand the uses and purposes of the disclosed devices, an exemplary deployment sequence is provided here. Referring to FIG. 1, a delivery system 1000 for the main body 100 and branch bodies 200 (see FIG. 3) is illustrated (not necessarily to scale). The delivery system 1000 is operable to deliver a multi-component implantable device (e.g., main body 100 and branch bodies 200) to a target site. The main body 100 includes a graft 102 supported by a frame 104. The delivery system includes a handle 1100, an elongate member 1200 having a first end 1202 coupled to and/or extending from the handle 1100 and a second end 1204, a cap 1300 positioned proximate the second end 1204 of the elongate member 1200, and at least one guide member 1400 extending at least partially along the elongate member 1200 toward the cap 1300. The elongate member 1200 and cap 1300 are operable to translate along a main guidewire 1500 (see FIG. 4). The delivery system 1000 can further include at least one sheath 1600 (see FIG. 6), the sheath 1600 operable for use with the guide member 1400 (when there are a plurality of guide members 1400a, 1400b, 1400c (see FIG. 5). An articulatable guide member 1700 (see FIGS. 6 and 7) can be included for each sheath 1600. The delivery system 1000 may also include a constraining member 1850 (see FIG. 5) that is operable to constrain at least a portion of a multi-component implantable device. It is understood that an individual constraining member 1850 may be implemented for each discrete component of the multi-component implantable device. The delivery system 1000 can be used in conjunction with filtration systems 2000 (e.g., to reduce risk of embolism, see FIG. 4).

FIG. 4 illustrates implementation of a main guidewire 1500 along which the delivery system 1000 may be implemented and a filtration system 2000 in connection with the delivery system 1000. The filtration system 2000 can include a plurality of deployable filters 2002 that can be deployed in discreet lumens, including side branch lumens, that are fluidically downstream from the target site at which the multi-component implantable device is to be implanted. The filtration system 2000 can be intermittently flushed throughout the procedure. A main guidewire 1500 is advanced to the target site (e.g., the aortic arch). Although the main guidewire 1500 is illustrated as coming from the descending aorta 23 (e.g., from a femoral access site), the main guidewire 1500 can be inserted from any appropriate access site.

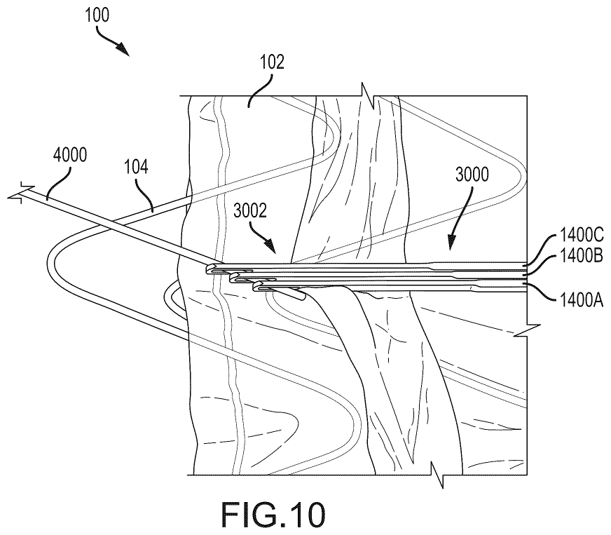

Referring to FIG. 5, a multi-component implantable device is advanced to the target site via the main guidewire 1500. For the purposes of the example provided herein, the multi-component implantable device will include the embodiment disclosed with respect to FIGS. 1 and 8. However, it is understood that the methods and the delivery system 1000 are not limited to delivering only the implantable device 10 as described with reference to FIGS. 1 and 3. The implantable device (e.g., the main body 100) is positioned on the elongate member 1200. For example, the implantable device 10 may be constrained in a compressed configuration about the elongate member 1200. For example, the implantable device 10 can be constrained by a constraining member 1850 (see FIG. 10). The implantable device 10 may be positioned proximate the cap 1300 and the second end 1204 of the elongate member 1200.

As is illustrated in FIG. 5, the delivery system 1000 may include a plurality of guide members 1400a, 1400b, 1400c, which in some embodiments are implemented to pre-cannulate side branch portals of the main body 100 to facilitate delivery of the side branches through the side branch portals into the side branches (e.g., a first branch body 200a in the brachiocephalic artery 24, a second branch body 200b in the left common carotid artery 25, and a third branch body 200c in the left subclavian artery 26). The guide members 1400a, 1400b, 1400c are coupled (e.g., releasably coupled) to the delivery system 1000 proximate the second end 1204 of the elongate member 1200. The present disclosure relates specifically to various systems and methods for releasably coupling guide members to the delivery system 1000 to assist in cannulation of branch vessels. However, prior to discussing each of the various embodiments in detail, the remainder of the exemplary procedure is discussed.

With further reference to FIG. 5, the guide members 1400a, 1400b, 1400c are each extending through a respective side branch portal 110 of the main body 100 of the implantable device. Cannulation of the side branch portals 110 occurs prior to insertion of the implantable device into the patient via the access site. Pre-cannulation can shorten the procedure time and simplify the steps performed during the operation, which can reduce trauma to the patient's tissue and damage to the implantable device. The guide members 1400a, 1400b, 1400c extend through the side branch portals 110 and through the second opening 109 of the main body 100 of the implantable device. Thus, the guide members 1400a, 1400b, 1400c may be positioned inside the main lumen 107of the implantable device from the side branch portals 110 to the second end 105 of the device. The guide members 1400a, 1400b, 1400c extend from the second opening 109 and toward the second end 1204 of the elongate member 1200. In some embodiments, the guide members 1400a, 1400b, 1400c are routed through the handle 1100 (see FIG. 1) and in other embodiments, the guide members 1400a, 1400b, 1400c are routed through other ports (not shown). The guide members 1400a, 1400b, 1400c may extend along the outside of the elongate member 1200, or the guide members 1400a, 1400b, 1400c may extend through the elongate member (not shown).

Referring now to FIG. 6, a sheath 1600 is provided for each guide member 1400. For example, when there is a first, second, and third guide member 1400a, 1400b, 1400c for the first, second, and third side branch portals 110a, 110b, 110c, a first, second, and third sheath (not shown) is provided for each corresponding branch body 200 and guide member 1400. Each sheath 1600 is operable to advance along each corresponding guide member 1400. The sheath 1600 can be formed to move along the guide member 1400 by surrounding the guide member 1400, by using the guide member as a rail in a side-by-side orientation, or as would otherwise permit the sheath 1600 the move substantially along the path of the guide member 1400. Because the side branch portals 110 are already pre-cannulated with the guide members 1400a, 1400b, 1400c, the sheath 1600 can be advanced through the second opening 109 of the elongate member and out the second opening 121 of the side branch portal 110.

For example, a first end 1602 of the sheath 1600 can be advanced through the vasculature of a patient and out the second opening 121 of the side branch portal 110. The first end 1602 may be positioned proximate the corresponding branch of the vasculature (e.g., the brachiocephalic artery).

In some embodiments, the sheath 1600 includes a lumen through which an articulatable guide member 1700 (e.g., a catheter) can be inserted (e.g., the same lumen through which the guide members 1400 are passed). Various secondary articulatable guide member 1700 may be implemented, including, but not limited to, steerable catheters and guidewires. For example, articulatable guide member 1700 can be steered using at least one tether or tension member (not shown) coupled to a distal end of the articulatable guide member 1700 (the articulatable guide member 1700 may be an integral unit, or may be a composite of various components for providing the articulating function, e.g., a guide catheter and a guidewire). The articulatable guide member 1700 may be steered by applying tension to the tether or tension member. Various degrees of motion can be achieved using multiple tethers and/or tension members. Other embodiments may include robotic or motor-driven guidewires. Various embodiments of an articulatable guidewire may be implemented in the delivery system 1000 and method. The articulatable guide member 1700 is advanced to the treatment site via the sheath 1600. For example, as illustrated in FIG. 6, a first articulatable guide member 1700a is advanced to the target site via the first sheath 1600a through first side branch portal 110. The articulatable guide member 1700 includes a leading end that can be articulated by the user at a trailing end (not shown). The leading end can bend or articulate to various configurations and positions. Once the leading end of the first articulatable guide member 1700a is free from the first sheath 1600a, the user is able to articulate the leading end of the first articulatable guide member 1700a into position in the target branch corresponding to the first side branch portal 110 (e.g., the brachiocephalic artery). Once the articulatable guide member 1700a is in place, a guidewire 1702 may be advanced into position (e.g., through the articulatable guide member 1700a). This step is repeated for each branch and corresponding side branch portal 110 with a subsequent articulatable guide member 1700 (see. FIG. 7).

In some embodiments, guide wires (not shown) may be advanced via the articulatable guide member 1700 which is then removed or the articulatable guide members 1700 may be used similar to a guide wire with respect to the branch bodies 200. The branch bodies 200 (FIG. 3) can be advanced along corresponding secondary, articulatable guide members 1700 (or guide wires that replate the articulatable guide members 1700). The branch bodies 200 can be advanced on independent components, for example, elongate members with end caps, and so forth, similar to the components used to deliver the main body 100. The end caps, or other independent components, can dilate the side branch portals 110 in some embodiments as the branch bodies 200 are passing through the side branch portals 110. The branch bodies 200 are positioned such that a first portion 202 is at least partially positioned in the branch of the target site and a second portion is positioned within the implantable device 10 (e.g., within the side branch portal 110). Once the branch bodies 200 are appropriately positioned, the branch bodies 200 are deployed.

Turning to a more specific discussion of the guide members 1400, the guide members 1400 are positioned to pre-cannulate the branches of the main body to facilitate cannulation of side branch vessels for bridging the main body device to the side branch vessels. FIGS. 8A-36D depict methods and systems to consistently and reliably attach and release guide members to serve as rail wires during deployment of a multibranch architecture device, such as implantable device 10. For discussion purposes, as is the case elsewhere herein, a first end of a component or similar refers to a proximal end of the component (e.g., proximal to the device for deployment at the target site). And, on the other hand, a second end of a component or similar refers to a distal end of the component (e.g., distal to the device for deployment at the target site such as at an operator or handle of the delivery system). It should be appreciated that many implementations of these principles can be employed in combination with or in alternative to features discussed elsewhere herein. For instance, guide members can include guidewires, guide catheters, or features of either discussed elsewhere herein.

In general, delivery system 1000 may include an elongate member 1200. The main body 100 is positioned about the elongate member 1200 (see FIG. 1). The main body 100 includes a main body wall 106 defining a main body lumen 107 and an access or opening to the side branch portal 110 through the main body wall 106 to provide access to the main body lumen 107 (see FIG. 1). The delivery system 1000 may include a plurality of guide members 1400 extending along at least a portion of the elongate member 1200, into the main body lumen 108, and through the side branch portal 110 (see also FIG. 1). The delivery system 1000 may include a guide member retainer assembly 2900 releasably engaging the guide member 1400 at a coupling portion such that the guide member 1400 is restricted from being retracted along the elongate member 1200 when the guide member 1400 is engaged with the guide member retainer assembly 2900.

Referring specifically to FIGS. 8A-36D, the delivery system 1000 according to principles of the present disclosure can include guide members 1400 for aiding and facilitating side branch cannulation for delivery of bridging or side branch stent grafts.

The guide members 1400 are selectively removable such that the guide members 1400 can be withdrawn from the delivery area or the patient's body after cannulation of the side branch vasculature is achieved. Various embodiments of how the guide members 1400 are releasably coupled to the proximal end of the delivery system for aiding in cannulation are provided in FIGS. 8A-36D. Broadly speaking, the guide member retainer assembly 2900 includes the guide member 1400, a guide member retainer 3000, and a release wire 4000. The guide member retainer 3000 and release element 4000 cooperate to removably couple the guide member 1400 to the proximal end of the delivery system 1000.

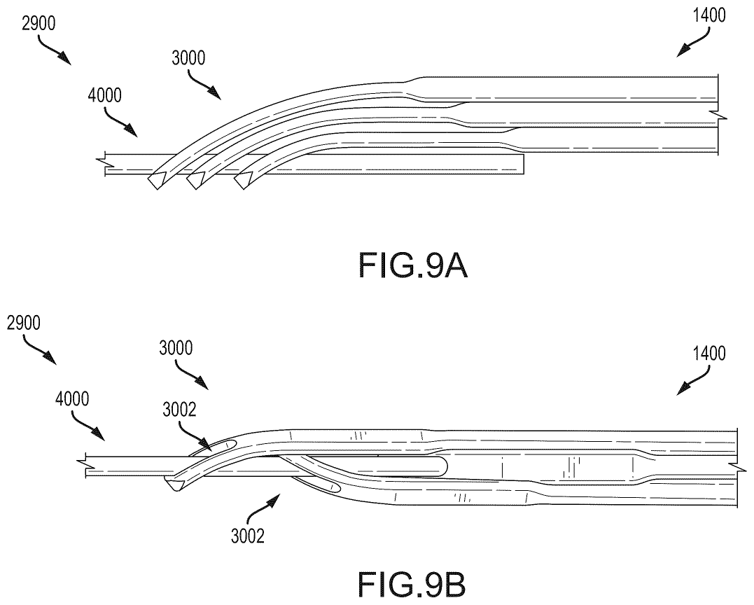

Referring specifically to FIGS. 8A-9B, in some embodiments the guide member retainer 3000 extends from or is positioned at a proximal end of the guide member 1400. The guide member retainer 3000 may comprise a structure with an eyelet 3002 defined therethrough. The structure may be an integral portion of the guide member 1400. In some embodiments, the guide member 1400 is a wire (e.g., a nitinol wire or any other type of appropriate wire) and the guide member retainer 3000 is positioned at the proximal end of the wire.

The retainer eyelet 3002 is defined at the proximal end of the guide member 1400, where the retainer eyelet 3002 defines an opening through which the release wire 4000 can be threaded. Various constructions of the retainer eyelet are contemplated herein and is not considered limiting. Several examples of forming the retainer eyelet 3002 include laser cutting a portion of the guide member retainer 3000), forming a loop by bending the proximal end of the guide member 1400 back upon itself and securing the overlapping portions together, such as through welding, crimping, or other suitable attachment methods, and so forth. The retainer eyelet 3002 may have a diameter that accommodates passage of the release wire 4000 while providing sufficient engagement to maintain the connection during delivery procedures.

The release wire 4000 extends through the retainer eyelet 3002 to secure the guide member 1400 in position relative to the delivery system 1000. When the release wire 4000 is positioned through the retainer eyelet 3002, the guide member 1400 is prevented from being withdrawn proximally beyond the position of the release wire 4000.

As shown in FIGS. 8A-8B, the guide member retainer 3000 may be configured in a substantially flat or planar arrangement. In this configuration, multiple guide members 1400 with their respective guide member retainers 3000 can be positioned adjacent to one another in a compact arrangement. The flat configuration may facilitate packaging and delivery of multiple guide members 1400 through a delivery catheter or sheath.

Alternatively, as illustrated in FIGS. 9A-9B, the guide member retainers 3000 may be arranged in a curved or nested configuration. In this arrangement, the guide member retainers 3000 may be provided with a precurved shape or a curved shape may be imparted on the retainers 300. The guide member retainers 3000 may be positioned at different longitudinal positions about the release wire 4000. The guide member retainers 3000 may be positioned relative to each other to distribute the guide members 1400 in a manner that corresponds to the longitudinal and/or angular positioning of the side branch portals 110 in the main body 100. The curved configuration may also help reduce the overall profile of the retainer assembly when multiple guide members 1400 are present. The retainers may include a thinner profile than a remainder of respective the guide members 1400. This may be accomplished by grinding a proximal end of the guide member 1400, flattening or pounding the proximal end of the guide member 1400, or by other means. The thinner profile may aid in a compact delivery profile.

The guide member retainer assembly 2900 allows for controlled release of the guide members 1400 by withdrawing the release wire 4000 from the retainer eyelets 3002. Once the release wire 4000 is withdrawn, the guide members 1400 are free to be removed from the delivery system 1000, allowing for withdrawal of the guide members 1400 from the patient as previously discussed.

FIG. 10 illustrates a guide member retainer assembly 2900 showing the interaction between a guide member retainer 3000, a release wire 4000, and components of the main body 100 including the graft 102 and the frame 104. The guide member retainer assembly 2900 may be configured to releasably couple guide members to the main body 100 of the implantable device.

In some cases, the guide member retainer 3000 may be coupled to the graft 102 of the main body 100 via the release wire 4000. The guide member retainer 3000 may include a retainer eyelet 3002 that is configured to engage with the release wire 4000. The release wire 4000 may pass through the graft 102 and the retainer eyelet 3002 to secure the guide member retainer 3000 to the main body 100.

The guide member retainer assembly 2900 may be used with multiple guide members. For example, a first guide member 1400a, a second guide member 1400b, and a third guide member 1400c may each be associated with a respective guide member retainer 3000. The guide member retainers 3000 may all simultaneously be released by withdrawing the release wire 4000. In some embodiments, each guide member retainer 3000 may be independently releasable from the main body 100 by withdrawing a respective release wire 4000 from the respective retainer eyelet 3002, where a release wire 4000 is provided for each guide member 1400.

In some embodiments, the graft 102 may limit withdrawal of the guide members 1400a, 1400b, 1400c beyond the position where the release wire 4000 passes through the graft 102. This configuration may allow for controlled release of the guide member retainer 3000 when the release wire 4000 is withdrawn from the retainer eyelet 3002.

These arrangements allow the guide members 1400a, 1400b, 1400c to serve as rail wires for guiding components of the implantable device during deployment.

The method of using the guide member retainer assembly 2900 may begin with positioning the guide members 1400 through the side branch portals 110 of the main body 100 in a pre-cannulated configuration. The guide member retainer 3000 may be positioned at the proximal end of each guide member 1400, with the retainer eyelet 3002 formed to accommodate the release wire 4000.

During assembly, the release wire 4000 may be threaded through the retainer eyelet 3002 of each guide member 1400. The release wire 4000 may pass through the graft 102 and engage with the retainer eyelets 3002 to secure the guide members 1400 in position relative to the delivery system 1000. This configuration may prevent the guide members 1400 from being withdrawn proximally beyond the position of the release wire 4000 during delivery and positioning procedures. In embodiments with multiple guide members, such as the first guide member 1400a, second guide member 1400b, and third guide member 1400c, the guide member retainers 3000 may be arranged in either a flat configuration or a curved, nested configuration along the release wire 4000. The curved configuration may allow the guide member retainers 3000 to be positioned at different longitudinal positions to correspond with the positioning of the side branch portals 110 in the main body 100.

The delivery system 1000 may then be advanced to the target site with the guide members 1400 pre-cannulating the side branch portals 110. Once the main body 100 is positioned at the target site, the guide members 1400 may serve as rail wires to facilitate cannulation of the side branch vessels. The pre-cannulated configuration may allow for more efficient and accurate positioning of subsequent components, such as sheaths 1600 and articulatable guide members 1700, through the side branch portals 110 and into the respective branch vessels.

When cannulation of the side branch vasculature is achieved and the guide members 1400 are no longer needed, the release wire 4000 may be withdrawn from the retainer eyelets 3002. This withdrawal may allow for controlled release of the guide member retainers 3000 from the main body 100. Once the release wire 4000 is withdrawn, the guide members 1400 may be free to be removed from the delivery system 1000 and withdrawn from the patient.

In embodiments where each guide member 1400 has an independent release wire 4000, the guide members 1400 may be released individually by withdrawing the respective release wire 4000 from each retainer eyelet 3002. This may allow for selective removal of guide members 1400 as different stages of the procedure are completed.

The graft 102 may provide a physical stop that limits withdrawal of the guide members 1400 beyond the position where the release wire 4000 passes through the graft 102, which may help ensure controlled and predictable release of the guide member retainer assembly 2900 during the procedure.

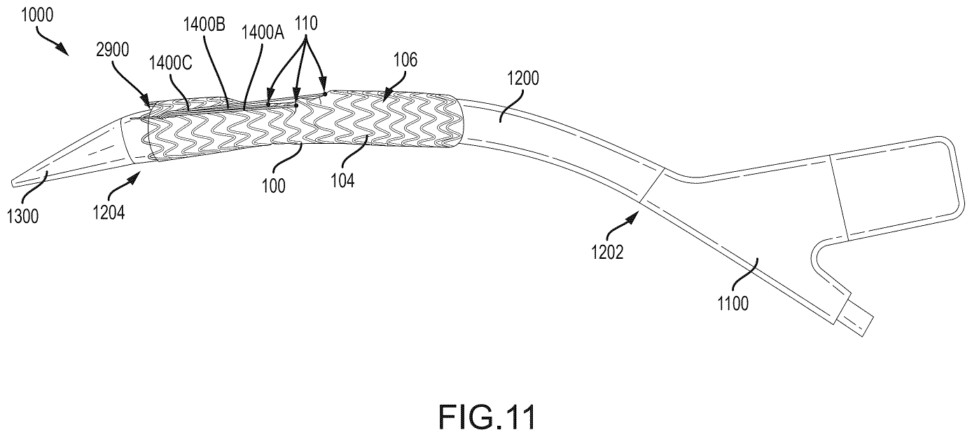

FIG. 11 illustrates a side view of the delivery system 1000 for deploying a medical device. The delivery system 1000 includes the elongate member 1200 extending between the first end 1202 and the second end 1204. The handle 1100 may be positioned at the first end 1202 (e.g., the distal end) of the elongate member 1200, while the cap 1300 may be located at the second end 1204 (e.g., the proximal end).

The delivery system 1000 may be configured to carry the graft 102 supported by the frame 104. The main body 100 may include the side branch portals 110 that are pre-cannulated with the guide members 1400. In some cases, the guide members 1400 may include the first guide member 1400a, the second guide member 1400b, and the third guide member 1400c.

A guide member retainer assembly 2900 may interface with the frame 104 beyond the side branch portals 110 (e.g., at a proximal end of the main body 100). This configuration may allow the guide members 1400 to be releasably coupled to the frame 104 via the guide member retainer assembly 2900. The arrangement may facilitate use of the guide members 1400 for navigating into side branch vasculature and then controlled release of the guide members 1400 during the deployment procedure after cannulation of the side branch vasculature through the side branch portals 110.

In some cases, the guide members 1400 may extend along at least a portion of the elongate member 1200, into a main lumen of the main body 100 (see FIG. 5), through the side branch portals 110, and to a proximal end of the main body 100. This configuration may allow the guide members 1400 to pre-cannulate the side branch portals 110, potentially simplifying the process of cannulating branching vessels during deployment.

The delivery system 1000 may be designed to allow for controlled deployment of the main body 100 and subsequent cannulation of branching vessels using the pre-cannulated guide members 1400. The releasable coupling of the guide members 1400 to the frame 104 via the guide member retainer assembly 2900 may provide a mechanism for selectively releasing the guide members 1400 when they are no longer needed in the deployment process.

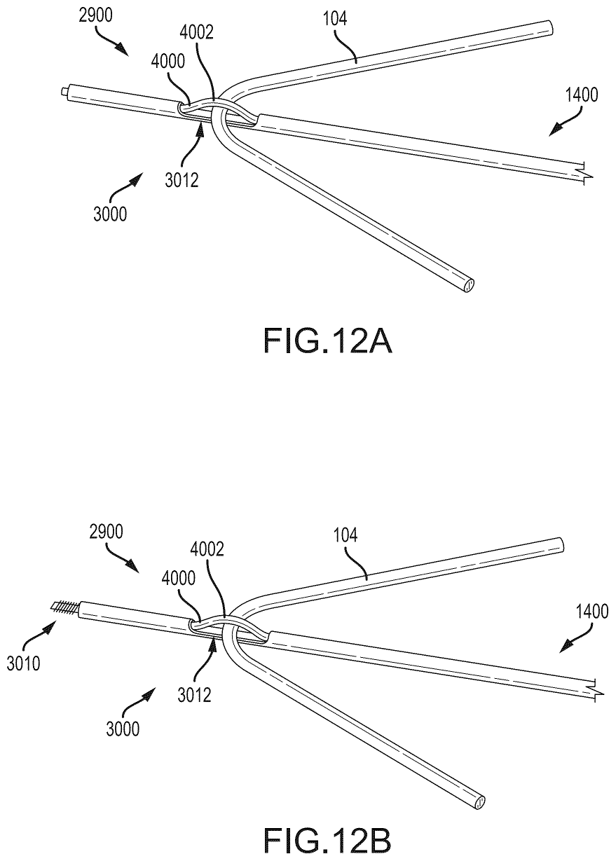

FIGS. 12A-12B illustrate orthogonal views of guide member retainer assemblies 2900. The guide member retainer assembly 2900 may include a guide member retainer 3000 that couples a guide member 1400 to a frame 104. The guide member retainer 3000 may be a portion of the guide member 1400 that defines a side wall opening 3012. A release wire 4000 is positioned through a lumen of the guide member 1400. The frame 104 may be positioned between the guide member 1400 and the release wire 4000 at the side wall opening 3012.

FIG. 12A shows the guide member retainer assembly 2900 in an engaged configuration. In this configuration, a curved portion 4002 of the release wire 4000 may extend through the side wall opening 3012 and back into the guide member 1400 where the frame 104 is positioned in a closed loop defined by the release wire 4000 and the guide member 1400. When the release wire 4000 is withdrawn through the guide member 1400, the closed loop is opened and the frame 104 is released from the guide member retainer assembly 2900.

The curved portion 4002 of the release wire 4000 may be shaped to facilitate engagement with the side wall opening 3012 and the frame 104. The curvature may allow for secure retention of the frame 104 while also permitting controlled release when the release wire 4000 is actuated.

In some cases, the side wall opening 3012 may be sized and shaped to accommodate the curved portion 4002 of the release wire 4000 and a portion of the frame 104. The dimensions of the side wall opening 3012 may be optimized to provide secure engagement while allowing for smooth release when desired.

FIG. 12B shows the guide member retainer assembly 2900 with threading 3010. The threading 3010 may further secure the release wire 4000 against premature actuation. In some cases, the threading 3010 may be defined on an inner surface of the guide member 1400 and an outer surface of the release wire 4000, which may provide additional resistance against unintended movement of the release wire 4000 until the threading is disengaged. The threading 3010 may provide an additional mechanism for controlling the movement of the release wire 4000. By engaging with the threading 3010, the release wire 4000 may require a deliberate rotational movement for actuation, which may reduce the risk of accidental release.

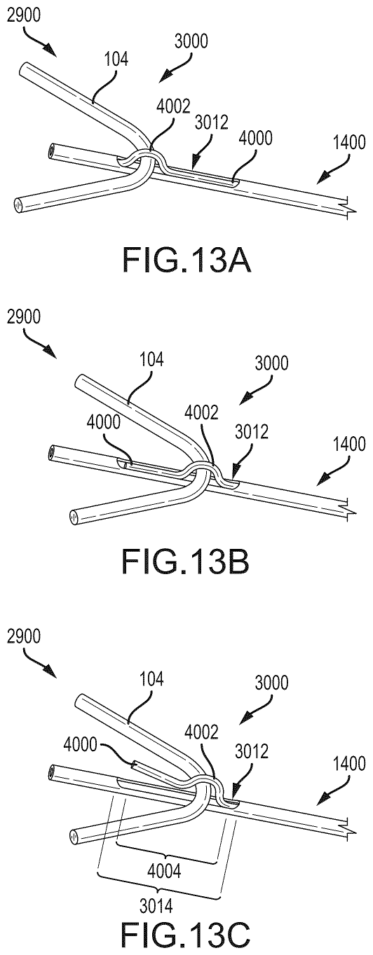

FIGS. 13A-13C illustrate orthogonal views of a guide member retainer assembly 2900 engaging with a frame 104. The guide member retainer assembly 2900 includes a guide member retainer 3000 having a side wall opening 3012. The release wire 4000 includes a curved portion 4002 that extends through the side wall opening 3012. The frame 104 may be positioned between the guide member 1400 and the release wire 4000 at the side wall opening 3012.

In some cases, the release mechanism of the guide member retainer assembly 2900 may utilize a long hypotube with an internal release wire. The guide member 1400 may comprise the hypotube, and the release wire 4000 may be positioned within a lumen of the hypotube. The curved portion 4002 of the release wire 4000 may extend out of the side wall opening 3012 in the hypotube to engage with the frame 104. As the release wire 4000 is withdrawn, the curved portion 4002 may translate through the side wall opening 3012, gradually disengaging from the frame 104.

The opening length 3014 of the side wall opening 3012 may be configured to allow for controlled translation of the curved portion 4002 during the release process. The release length 4004 may correspond to the length of the release wire 4000 that needs to be withdrawn to fully disengage the curved portion 4002 from the frame 104.

FIG. 13A shows a guide member retainer assembly 2900 with the release wire 4000 engaged with the guide member retainer 3000. The side wall opening 3012 has an opening length 3014 that may accommodate the curved portion 4002 of the release wire 4000 plus additional length of the release wire 4000. Stated otherwise, the curved portion 4002 has a shorter longitudinal length than the side wall opening.

FIG. 13B depicts the guide member retainer assembly 2900 during an intermediate stage of release. The release wire 4000 may slide relative to the guide member 1400 such that the curved portion 4002 of the release wire 4000 begins to translate within the side wall opening 3012.

FIG. 13C shows the guide member retainer assembly 2900 in a further stage of release. The curved portion 4002 of the release wire 4000 may continue to withdraw along the side wall opening 3012 of the guide member retainer 3000. The release wire 4000 may translate relative to the guide member 1400 such that the curved portion 4002 of the release wire 4000 has translated to a distal end of the side wall opening 3012. A release length 4004 of the release wire 4000 may be freed from the guide member retainer 3000 of the guide member 1400, which releases the proximal end of the release wire 4000 and consequently may free the frame 104 from the guide member retainer assembly 2900 and allow the guide member 1400 to be withdrawn from the frame 104.

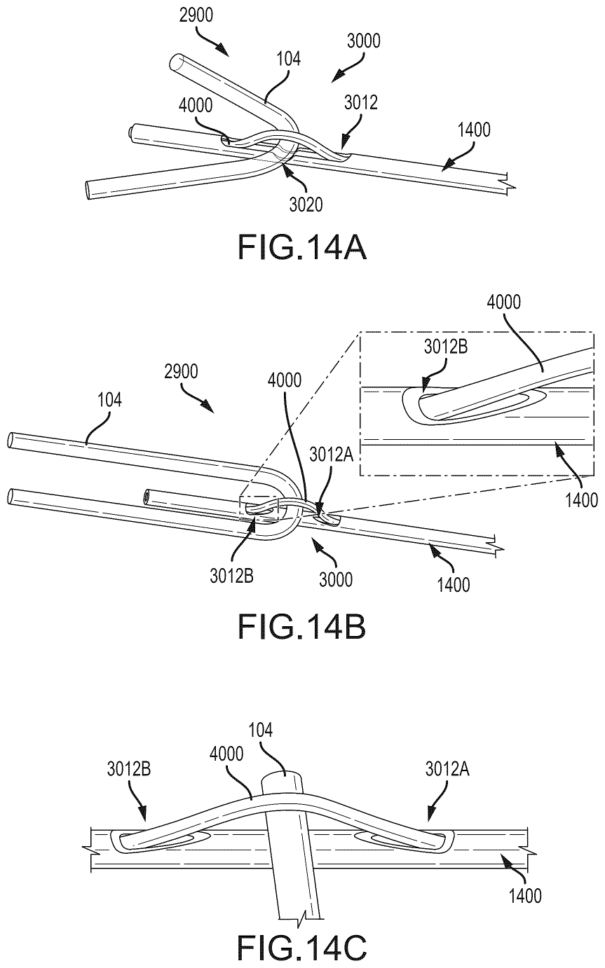

FIGS. 14A-14C and FIG. 16 illustrate various views of guide member retainer assemblies 2900 for securing a guide member 1400 to a frame 104.

FIG. 14A shows a side view of the guide member retainer assembly 2900 including a guide member retainer 3000 having a side wall opening 3012 through which a release wire 4000 passes. The guide member retainer 3000 includes a frame retainer groove 3020 configured to engage with the frame 104. In some cases, the frame retainer groove 3020 may reduce the profile of the guide member retainer assembly 2900. The frame retainer groove 3020 may limit movement of the frame 104 relative to the guide member retainer assembly 2900 when engaged. Additionally, the frame retainer groove 3020 facilitates a compact design by reducing the distance the release wire 4000 extends out of the side wall opening 3012.

FIG. 14B depicts another side view of the guide member retainer assembly 2900, showing a first side wall opening 3012A and a second side wall opening 3012B in the guide member retainer 3000. The release wire 4000 may extend through both side wall openings to secure the guide member retainer 3000 to the frame 104. Specifically, the release wire 4000 extends out of the first side wall opening 3012A and then back into the guide member 1400 through the second side wall opening 3012B. In some cases, the first side wall opening 3012A and the second side wall opening 3012B may provide additional points of engagement for the release wire 4000, potentially enhancing the security of the release wire 4000 to limit accidental engagement of the release wire 4000.

FIG. 14C provides a magnified view of the guide member retainer assembly 2900, particularly illustrating the interaction between the first side wall opening 3012A and the second side wall opening 3012B with the release wire 4000. The guide member retainer assembly 2900 may engage with the frame 104, while the guide member 1400 extends through the assembly.

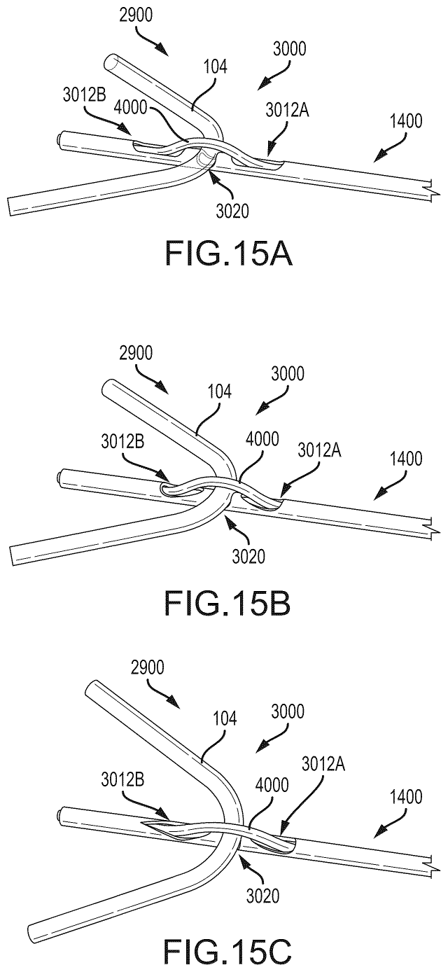

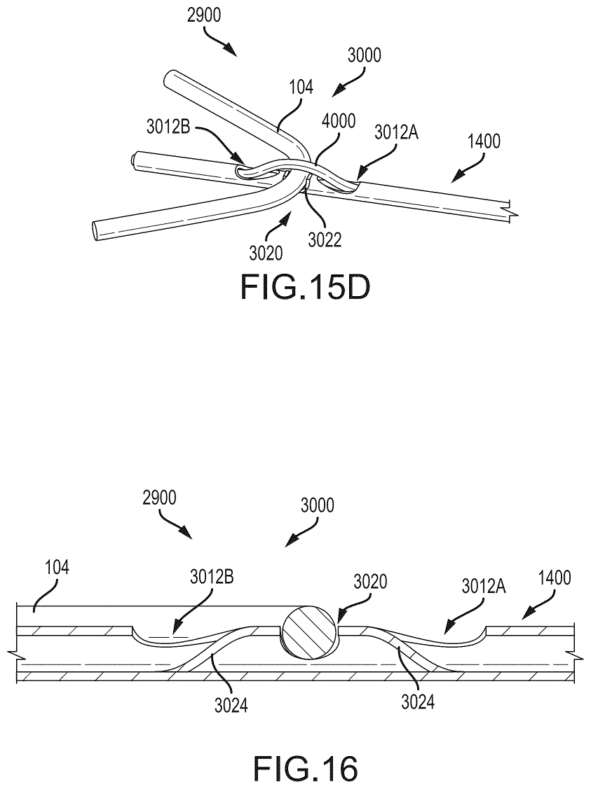

FIGS. 15A-15D illustrate orthogonal views of guide member retainer assemblies 2900 showing different configurations and interactions between components.

In FIG. 15A, the guide member retainer assembly 2900 may be coupled to the frame 104. The assembly may include a guide member retainer 3000 having a first side wall opening 3012A and a second side wall opening 3012B. The release wire 4000 extend through the guide member 1400, out the first side wall opening 3012A, and back into the second side wall opening 3012B. The guide member retainer 3000 may include the frame retainer groove 3020 that engages with the frame 104.

FIG. 15B depicts the guide member retainer assembly 2900 with the release wire 4000 positioned to secure the guide member 1400 through the first side wall opening 3012A and the second side wall opening 3012B. The frame retainer groove 3020 may maintain engagement with the frame 104. In some cases, the first side wall opening 3012A and the second side wall opening 3012B may have a different shape as compared to FIG. 15A. The various shapes may provide various benefits such as smoother translation through the openings or increased resistance to translation of the release wire 4000 relative to the guide member 1400.

FIG. 15C depicts the guide member retainer assembly 2900 with the release wire 4000 positioned to secure the guide member 1400 through the first side wall opening 3012A and the second side wall opening 3012B. The frame retainer groove 3020 may maintain engagement with the frame 104. In some cases, the first side wall opening 3012A and the second side wall opening 3012B may have a different shape as compared to FIGS. 15A and 15B. Additionally, the first side wall opening 3012A and the second side wall opening 3012B may have different shapes from each other. In some cases, the second side wall opening 3012B (or the first side wall opening 3012A) may include an elongated slit or sharp notch for locking the release wire 4000 from release when movements other than withdrawal of the release wire 4000 occur.

FIG. 15D illustrates the guide member retainer assembly 2900 with a reinforcing member 3022 positioned near the frame retainer groove 3020. The reinforcing member 3022 may provide additional structure around the area where the guide member retainer 3000 may be coupled to the frame 104. The release wire 4000 and the guide member 1400 may be shown in their assembled configuration with the first side wall opening 3012A and the second side wall opening 3012B.

The variations in the configurations of the first side wall opening 3012A and the second side wall opening 3012B across FIGS. 15A-15D may demonstrate different approaches to securing the release wire 4000 and controlling its movement. These variations may allow for adjustments in the release mechanism to suit different deployment scenarios or to provide varying levels of security against unintended release.

FIG. 16 illustrates a section view of a guide member retainer assembly 2900 engaged with the frame 104. The guide member retainer 3000 includes a first side wall opening 3012A and a second side wall opening 3012B positioned along the length of the guide member retainer 3000. The frame retainer groove 3020 may be formed in the guide member retainer 3000 between the first and second side wall openings 3012A, 3012B. In some cases, the frame retainer groove 3020 may be configured to receive a portion of the frame 104.

The guide member retainer assembly 2900 may include tabs 3024 extending from the guide member retainer 3000. The tabs 3024 may be positioned to provide a ramp and smooth surface along which the release wire 4000 translates during both assembly and during withdrawal as part of the delivery. In some cases, the tabs 3024 may facilitate smoother movement of the release wire 4000 and potentially reduce the risk of snagging or catching during deployment or withdrawal.

The configuration of the frame retainer groove 3020 and the first and second side wall openings 3012A, 3012B may allow for secure engagement of the guide member 1400 with the frame 104 while also providing a mechanism for controlled release. When the release wire 4000 is withdrawn, the frame 104 may be freed from the guide member retainer assembly 2900, allowing for removal of the guide member 1400 after use in cannulating side branch vessels.

The configuration of the frame retainer groove 3020 and the first and second side wall openings 3012A, 3012B may allow for secure engagement of the guide member 1400 with the frame 104 while also providing a mechanism for controlled release. When the release wire 4000 is withdrawn, the frame 104 may be freed from the guide member retainer assembly 2900, allowing for removal of the guide member 1400 after use in cannulating side branch vessels.

It should be appreciated that the various features illustrated in FIGS. 12A-14C and FIG. 16, including the frame retainer groove 3020, side wall opening 3012 or first and second side wall openings 3012A, 3012B, reinforcing member 3022, and tabs 3024, may be implemented in various combinations with one another including various shapes and combinations of shapes. The disclosure is not limited to the specific combinations discussed or illustrated in these figures. In some aspects, any combination of these features may be utilized to achieve the desired retention and release characteristics for the guide member retainer assembly 2900. The selection of particular feature combinations may depend on factors such as the specific application requirements, manufacturing considerations, or performance objectives for the delivery system 1000.

The method of using the guide member retainer assembly 2900 of FIGS. 11-16 may begin with positioning the guide members 1400 through the side branch portals 110 of the main body 100 in a pre-cannulated configuration. The guide member retainer 3000 may be positioned at the proximal end of each guide member 1400, with the side wall opening 3012 or first and second side wall openings 3012A, 3012B formed to accommodate the release wire 4000.

During assembly, the release wire 4000 may be threaded through the guide member 1400 and positioned to extend through the side wall opening 3012. In configurations with first and second side wall openings 3012A, 3012B, the release wire 4000 may extend out of the first side wall opening 3012A and back into the guide member 1400 through the second side wall opening 3012B. The curved portion 4002 of the release wire 4000 may extend through the side wall opening 3012 to engage with the frame 104. This configuration may prevent the guide members 1400 from being withdrawn proximally beyond the position of the interface between the frame 104 and the release wire 4000 during delivery and positioning procedures.

In embodiments with multiple guide members, such as the first guide member 1400a, second guide member 1400b, and third guide member 1400c, the guide member retainers 3000 may be arranged to correspond with the positioning of the side branch portals 110 in the main body 100. The frame retainer groove 3020 may engage with the frame 104 to provide secure coupling during delivery.

The delivery system 1000 may then be advanced to the target site with the guide members 1400 pre-cannulating the side branch portals 110. Once the main body 100 is positioned at the target site, the guide members 1400 may serve as rail wires to facilitate cannulation of the side branch vessels. The pre-cannulated configuration may allow for more efficient and accurate positioning of subsequent components, such as sheaths 1600 and articulatable guide members 1700, through the side branch portals 110 and into the respective branch vessels.

When cannulation of the side branch vasculature is achieved and the guide members 1400 are no longer needed, the release wire 4000 may be withdrawn from the guide member 1400. This withdrawal may cause the release wire 400 to translate through the side wall opening 3012, freeing a proximal end of the release wire 400 allowing the guide member 1400 to disengage from the frame 104. The opening length 3014 of the side wall opening 3012 may allow for controlled translation of the curved portion 4002 during the release process.

As the release wire 4000 continues to be withdrawn, the curved portion 4002 may translate to a distal end of the side wall opening 3012. A release length 4004 of the release wire 4000 may be freed from the guide member retainer 3000, which may release the proximal end of the release wire 4000 and consequently free the frame 104 from the guide member retainer assembly 2900. Once the frame 104 is released, the guide member 1400 may be withdrawn from the delivery system 1000 and removed from the patient.

In embodiments where each guide member 1400 has an independent release wire 4000, the guide members 1400 may be released individually by withdrawing the respective release wire 4000 from each guide member retainer 3000. This may allow for selective removal of guide members 1400 as different stages of the procedure are completed.

The tabs 3024 may facilitate smoother movement of the release wire 4000 during withdrawal, potentially reducing the risk of snagging or catching during the release process. The frame retainer groove 3020 may provide a physical stop that helps ensure controlled and predictable release of the guide member retainer assembly 2900 during the procedure.

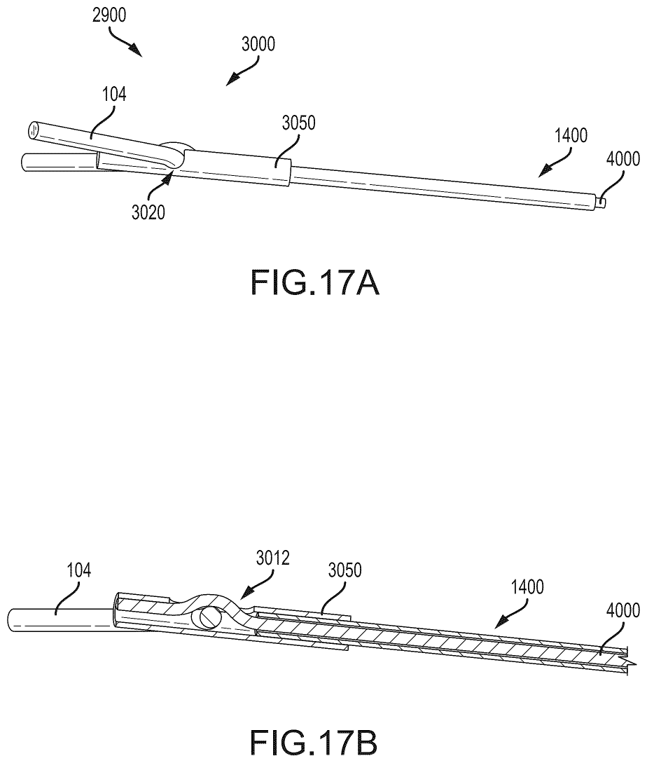

FIGS. 17A-17B illustrate orthogonal views of a guide member retainer assembly 2900. The guide member retainer assembly 2900 may include a guide member 1400 coupled to a proximal hypotube 3050. The proximal hypotube 3050 may include a side wall opening 3012 and a frame retainer groove 3020. The frame retainer groove 3020 may be formed in the proximal hypotube 3050 to engage with the frame 104.

A release wire 4000 may be positioned within the proximal hypotube 3050 such that the frame 104 may be retained between the release wire 4000 and the proximal hypotube 3050. In some cases, the release wire 4000 may be actuated to selectively release the frame 104 from engagement with the guide member retainer 3000. The proximal hypotube 3050 may be implemented to provide additional strength to the guide member retainer assembly 2900 while allowing the majority of the length of the guide member 1400 to include a minimal profile.

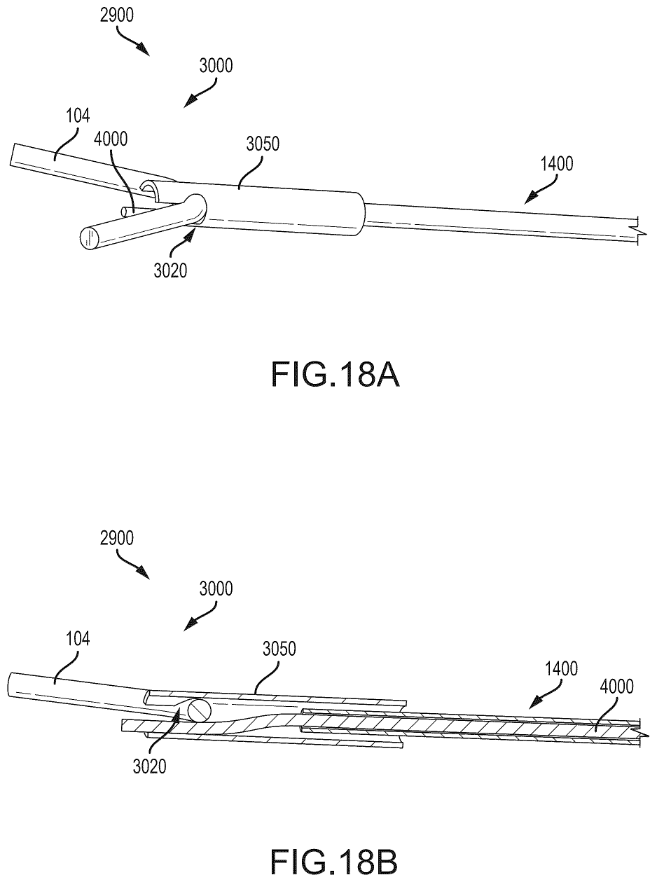

FIGS. 18A-18B illustrate additional orthogonal and section views of a guide member retainer assembly 2900. In FIG. 18A, the guide member retainer assembly 2900 may include a proximal hypotube 3050 coupled to the guide member 1400. The proximal hypotube 3050 may include a frame retainer groove 3020 within which the frame 104 may be positioned.

In some cases, the frame retainer groove 3020 may include a lip which may provide mechanical interference to movement of the frame 104 relative to the guide member retainer 3000 when the release wire 4000 may be positioned therewith. When the release wire 4000 may be withdrawn, the guide member retainer 3000 may provide sufficient clearance around the frame retainer groove 3020 for removal of the frame 104 from the guide member retainer 3000.

FIG. 18B shows a section view of the guide member retainer assembly 2900 of FIG. 18A, depicting how the guide member retainer 3000 may engage with the frame 104. The proximal hypotube 3050 is coupled to the proximal end of the guide member 1400, with the release wire 4000 extending through the guide member 1400 and positioned within the proximal hypotube 3050. The frame retainer groove 3020 of the guide member retainer 3000 may interface with the frame 104, illustrating the engagement mechanism between these components. When the release wire 4000 is positioned through the proximal hypotube 3050, the frame is restricted to a position within the frame retainer groove 3020 as there is insufficient room within the proximal hypotube 3050 when the release wire 4000 is also positioned therein. Removal of the release wire 4000 opens additional space to allow the frame 104 to disengage from the retainer groove and to allow separation of guide member 1400 from the frame 104.

The method of using the guide member retainer assembly 2900 of FIGS. 17A-18B may begin with positioning the guide members 1400 through the side branch portals 110 of the main body 100 in a pre-cannulated configuration. The proximal hypotube 3050 may be coupled to the proximal end of each guide member 1400, with the frame retainer groove 3020 formed to accommodate the frame 104.

During assembly, the release wire 4000 may be positioned within the proximal hypotube 3050 such that the frame 104 is retained between the release wire 4000 and the proximal hypotube 3050. The frame retainer groove 3020 may engage with the frame 104 to provide secure coupling during delivery, with the lip of the frame retainer groove 3020 providing mechanical interference to prevent unintended movement of the frame 104.

The delivery system 1000 may then be advanced to the target site with the guide members 1400 pre-cannulating the side branch portals 110. Once the main body 100 is positioned at the target site, the guide members 1400 may serve as rail wires to facilitate cannulation of the side branch vessels.

When cannulation of the side branch vasculature is achieved and the guide members 1400 are no longer needed, the release wire 4000 may be withdrawn from the proximal hypotube 3050. This withdrawal may create additional space within the proximal hypotube 3050, allowing the frame 104 to disengage from the frame retainer groove 3020. Once the frame 104 is released from the retainer groove 3020, the guide member 1400 may be separated from the frame 104 and withdrawn from the delivery system 1000 and removed from the patient.

The proximal hypotube 3050 may provide additional strength to the guide member retainer assembly 2900 during the procedure while maintaining a minimal profile for the majority of the guide member 1400 length, which may facilitate smooth withdrawal through the patient's vasculature.

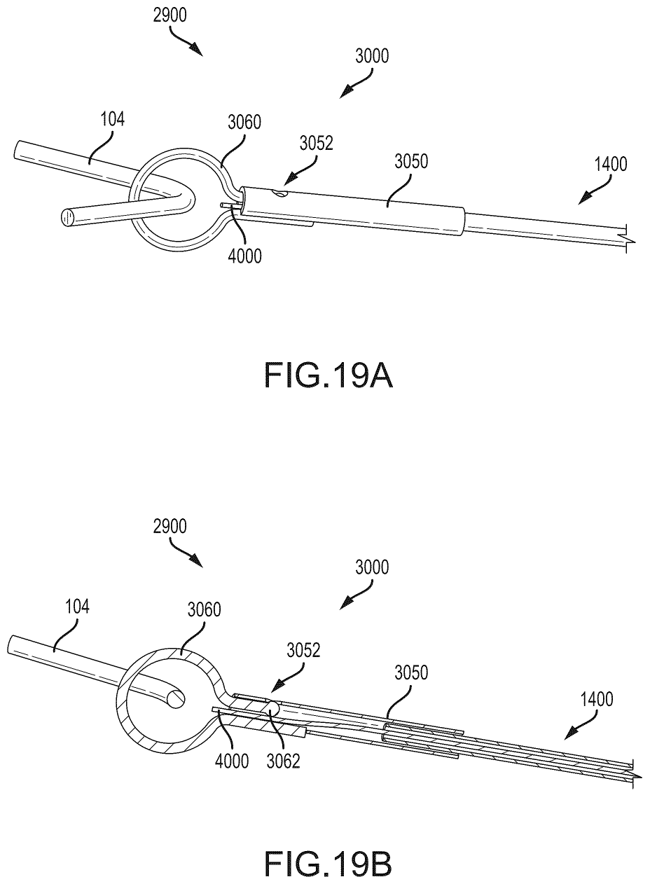

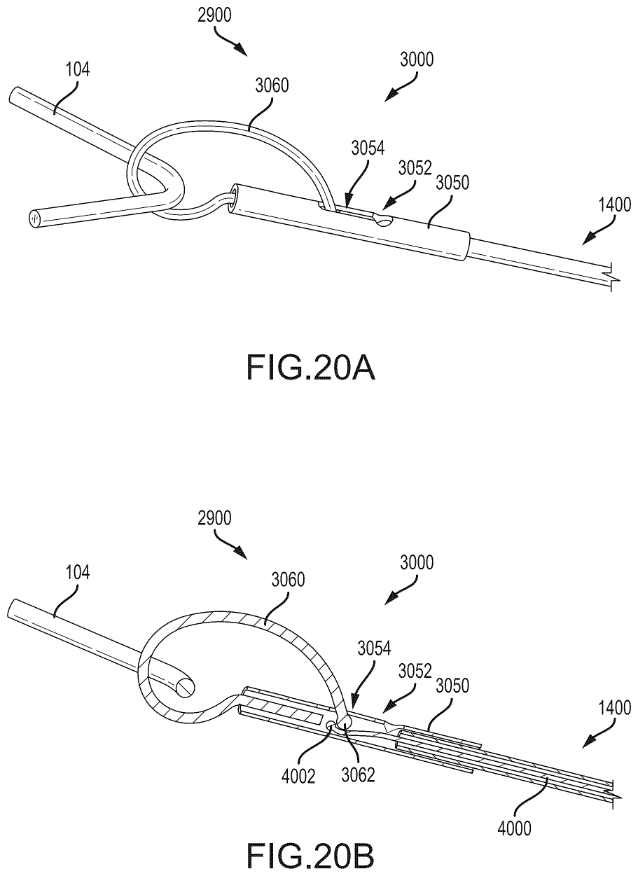

FIGS. 19A-19B and FIGS. 20A-20B illustrate orthogonal and section views of a guide member retainer assembly 2900. The guide member retainer assembly 2900 may include the guide member 1400 releasably coupled to the frame 104.

In some cases, the proximal hypotube 3050 may be coupled to the guide member 1400. A loop 3060 may be coupled to the proximal hypotube 3050. For example, one end of the loop 3060 may be welded to an exterior surface of the proximal hypotube 3050 and the other end may be positioned in a lumen of the proximal hypotube 3050.

The loop 3060 may include a retainer ball 3062 and the proximal hypotube 3050 may include a sidewall aperture 3052 within which the retainer ball 3062 may be seated. The release wire 4000 may extend through the proximal hypotube 3050 and engage with the retainer ball 3062 when the retainer ball 3062 is seated in the sidewall aperture 3052 such that the second end of the loop 3060 may be immobile when the release wire 4000 is engaged. When the release wire 4000 is withdrawn, the lumen of the proximal hypotube 3050 may have sufficient clearance for the retainer ball 3062 to disengage from the proximal hypotube 3050 thus allowing the guide member retainer assembly 2900 to disengage from the frame 104.

In some cases, as illustrated in FIGS. 20A-20B, one end of the loop 3060 may be welded to an interior surface of the proximal hypotube 3050, extend through a proximal opening of the proximal hypotube 3050, and reenter the proximal hypotube 3050 through the sidewall aperture 3052. The proximal hypotube 3050 may include the sidewall aperture 3052 and a slot 3054. The slot 3054 may be connected to the sidewall aperture 3052, where the slot 3054 may be narrower than the retainer ball 3062 and the sidewall aperture 3052 may be wider than the retainer ball 3062.

When the retainer ball 3062 is positioned proximate the slot 3054, the retainer ball 3062 may be unable to disengage from the proximal hypotube 3050. When the retainer ball 3062 is positioned proximate the sidewall aperture 3052, the retainer ball 3062 may travel through the sidewall aperture 3052 and disengage from the proximal hypotube 3050.

The release wire 4000 may extend through the proximal hypotube 3050 and engage with the retainer ball 3062. The release wire 4000 may include a laser ball 4001 positioned proximal the retainer ball 3062. When the release wire 4000 is withdrawn, the laser ball 4001 translates the retainer ball 3062 along the slot 3054 toward the sidewall aperture 3052 such that the retainer ball 3062 may be withdrawn through the sidewall aperture 3052. When the retainer ball 3062 is withdrawn through the sidewall aperture 3052, the guide member retainer assembly 2900 may be disengaged from the frame 104.

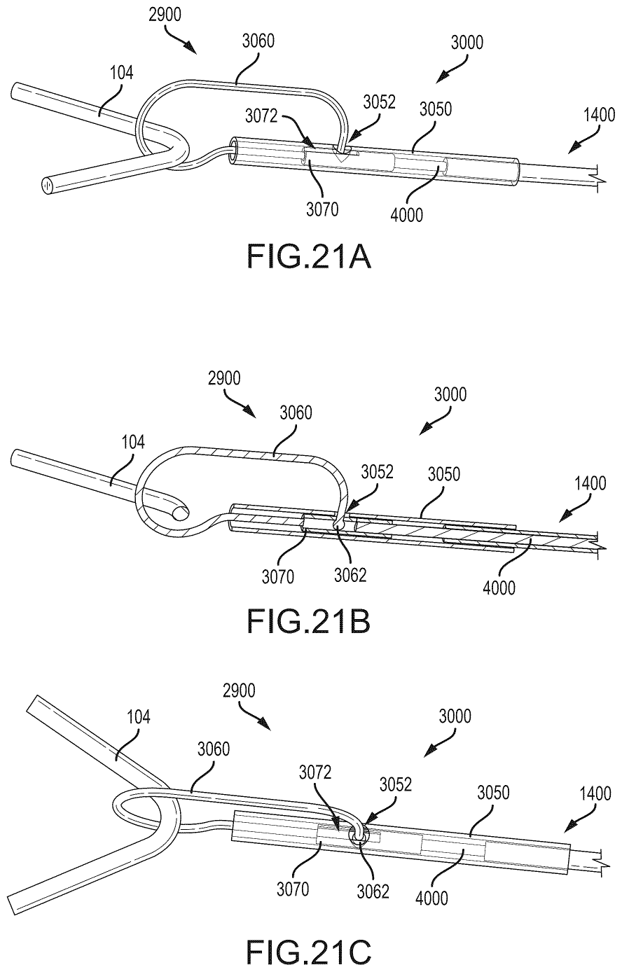

FIGS. 21A-21C illustrate various views of a guide member retainer assembly 2900. The guide member retainer assembly 2900 includes a guide member 1400 that is coupled to the frame 104. The guide member retainer 3000 may include a proximal hypotube 3050 having a sidewall aperture 3052. A loop 3060 may extend from the proximal hypotube 3050, and the retainer ball 3062 may be positioned at an end of the loop 3060.

The assembly may include a second proximal hypotube 3070 positioned within a lumen of the proximal hypotube 3050. The second proximal hypotube 3070 may feature a slot 3072 positioned near the sidewall aperture 3052 of the proximal hypotube 3050. In some cases, the loop 3060 may extend through the sidewall aperture 3052 and the slot 3072. The slot 3072 may be narrower than the retainer ball 3062 of the loop 3060 to restrict release of the retainer ball 3062. The sidewall aperture 3052 may be oversized relative to the retainer ball 3062 of the loop 3060 to allow the retainer ball 3062 of the loop 3060 to pass therethrough.

The release wire 4000 may extend through the proximal hypotube 3050 and may be coupled to the second proximal hypotube 3070. When the release wire 4000 is withdrawn, the second proximal hypotube 3070 translates within the lumen of the proximal hypotube 3050. When the second proximal hypotube 3070 translates within the lumen of the proximal hypotube 3050, the retainer ball 3062 of the loop 3060 may be pulled through the sidewall aperture 3052 and released from the second proximal hypotube 3070.

The method of using the guide member retainer assembly 2900 of FIGS. 19A-21C may begin with positioning the guide members 1400 through the side branch portals 110 of the main body 100 in a pre-cannulated configuration. The proximal hypotube 3050 may be coupled to the proximal end of each guide member 1400, with the loop 3060 and retainer ball 3062 positioned to engage with the sidewall aperture 3052 and to engage with the frame 104.

During assembly, the release wire 4000 may be positioned within the proximal hypotube 3050 to engage with the retainer ball 3062. In configurations with the laser ball 4001, the laser ball 4001 may be positioned proximal to the retainer ball 3062 to facilitate controlled movement along the slot 3054. The retainer ball 3062 may be seated within the sidewall aperture 3052, with the slot 3054 providing a narrower pathway that restricts unintended release.

The delivery system 1000 may then be advanced to the target site with the guide members 1400 pre-cannulating the side branch portals 110. Once the main body 100 is positioned at the target site, the guide members 1400 may serve as rail wires to facilitate cannulation of the side branch vessels.

When cannulation of the side branch vasculature is achieved and the guide members 1400 are no longer needed, the release wire 4000 may be withdrawn from the proximal hypotube 3050. In embodiments with the laser ball 4001, withdrawal of the release wire 4000 may cause the laser ball 4001 to translate the retainer ball 3062 along the slot 3054 toward the sidewall aperture 3052. Once the retainer ball 3062 reaches the sidewall aperture 3052, it may pass through the aperture and disengage from the proximal hypotube 3050.

In configurations with the second proximal hypotube 3070, withdrawal of the release wire 4000 may cause the second proximal hypotube 3070 to translate within the lumen of the proximal hypotube 3050. This translation may allow the retainer ball 3062 to be pulled through the sidewall aperture 3052 and released from the second proximal hypotube 3070.

Once the retainer ball 3062 is released, the guide member retainer assembly 2900 may disengage from the frame 104, allowing the guide member 1400 to be withdrawn from the delivery system 1000 and removed from the patient. The loop 3060 configuration may provide secure retention during delivery while allowing for controlled release when desired.

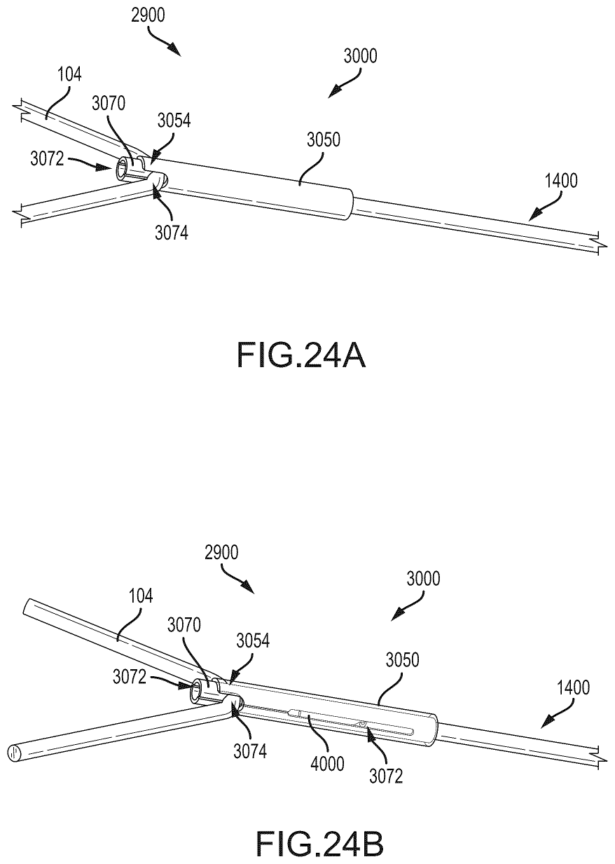

FIGS. 24A-24B illustrate orthogonal and partial section views of a guide member retainer assembly 2900. The guide member retainer assembly 2900 may include a guide member 1400 and a guide member retainer 3000. The guide member retainer 3000 may comprise a proximal hypotube 3050 having a slot 3054 and positioned longitudinally moveable about a portion of the guide member 1400.

The guide member 1400 may be coupled to a second proximal hypotube 3070 that may include a retainer 3074 and the slot 3072. The proximal hypotube 3050 may be positioned about the second proximal hypotube 3070 and may translate relative to the second proximal hypotube 3070 and the guide member 1400.

The release wire 4000 extends through the guide member 1400 and out the slot 3072 of the second proximal hypotube 3070 and is coupled to an interior surface of the proximal hypotube 3050. The slot 3072 may extend to the proximal end of the second proximal hypotube 3070 thus allowing the proximal end of the second proximal hypotube 3070 to splay open to release the frame 104.

The frame 104 is positioned through the retainer 3074 of the second proximal hypotube 3070 and the slot 3054 of the proximal hypotube 3050 such that the frame 104 is retained by the retainer 3074. The proximal hypotube 3050 covers and supports the second proximal hypotube 3070 to restrict the second proximal hypotube 3070 from splaying upon resulting in release of the frame 104 from the guide member retainer 3000. As the release wire 4000 is withdrawn, the proximal hypotube 3050 translates away from the frame 104 to allow the proximal end of the second proximal hypotube 3070 to splay open thus allowing the frame 104 to be freed from the guide member retainer 3000.

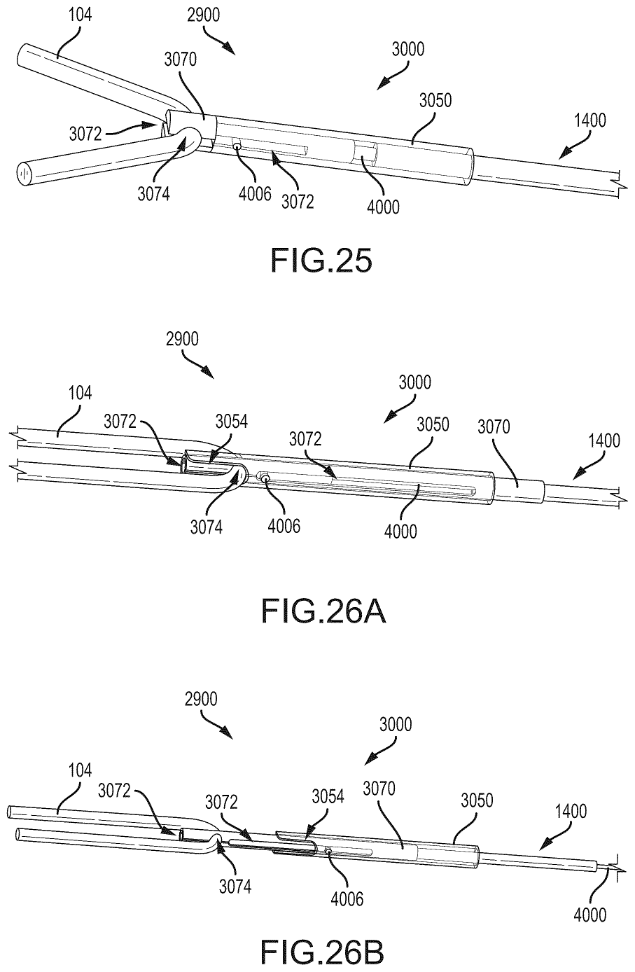

FIG. 25 illustrates an orthogonal view of a guide member retainer assembly 2900. The guide member retainer assembly 2900 may include a guide member 1400 and a guide member retainer 3000. The guide member retainer 3000 may comprise a proximal hypotube 3050 positioned longitudinally moveable about a portion of the guide member 1400.

The guide member 1400 may be coupled to the second proximal hypotube 3070 that includes a retainer 3074 and a slot 3072. The proximal hypotube 3050 may be positioned about the second proximal hypotube 3070 and may translate relative to the second proximal hypotube 3070 and the guide member 1400.

The release wire 4000 may extend through the guide member 1400 and may be coupled to a pin 4006 at a proximal end. The pin 4006 may be coupled to an interior surface of the proximal hypotube 3050 through the slot 3072 of the second proximal hypotube 3070. The slot 3072 extends longitudinally such that the pin 4006 can travel longitudinally within the slot 3072. The slot 3072 may extend to the proximal end of the second proximal hypotube 3070 thus allowing the proximal end of the second proximal hypotube 3070 to splay open to release the frame 104.

The frame 104 may be positioned through the retainer 3074 of the second proximal hypotube 3070 and the slot 3054 of the proximal hypotube 3050 such that the frame 104 may be covered by the proximal hypotube 3050 to restrict removal of the frame 104 from the guide member retainer 3000. As the release wire 4000 is withdrawn, the proximal hypotube 3050 may translate away from the frame 104 (the pin 4006 travels along a length of the slot 3072 and pulls the proximal hypotube 3050) to allow the proximal end of the second proximal hypotube 3070 to splay open thus allowing the frame 104 to be freed from the guide member retainer 3000.

FIGS. 26A-26B illustrate orthogonal views of a guide member retainer assembly 2900. The guide member retainer assembly 2900 may include a guide member 1400 and a guide member retainer 3000. The guide member retainer 3000 may comprise a proximal hypotube 3050 positioned longitudinally moveable about a portion of the guide member 1400.

The guide member 1400 may be coupled to a second proximal hypotube 3070 that may include a retainer 3074 and a slot 3072. The proximal hypotube 3050 may be positioned about the second proximal hypotube 3070 and may cover the second proximal hypotube 3070 beyond the retainer 3074. The proximal hypotube 3050 may translate relative to the second proximal hypotube 3070 and the guide member 1400.

The release wire 4000 may extend through the guide member 1400 and may be coupled to the pin 4006 at a proximal end. The pin 4006 may be coupled to an interior surface of the proximal hypotube 3050. The slot 3072 extends longitudinally such that the pin 4006 can travel longitudinally within the slot 3072. The slot 3072 may extend to the proximal end of the second proximal hypotube 3070 thus allowing the proximal end of the second proximal hypotube 3070 to splay open to release the frame 104.

The frame 104 may be positioned through the retainer 3074 of the second proximal hypotube 3070 and the slot 3054 of the proximal hypotube 3050 such that the frame 104 may be covered by the proximal hypotube 3050 to restrict removal of the frame 104 from the guide member retainer 3000. As the release wire 4000 is withdrawn, the proximal hypotube 3050 may translate away from the frame 104 (the pin 4006 travels along a length of the slot 3072 and pulls the proximal hypotube 3050) to allow the proximal end of the second proximal hypotube 3070 to splay open thus allowing the frame 104 to be freed from the guide member retainer 3000.

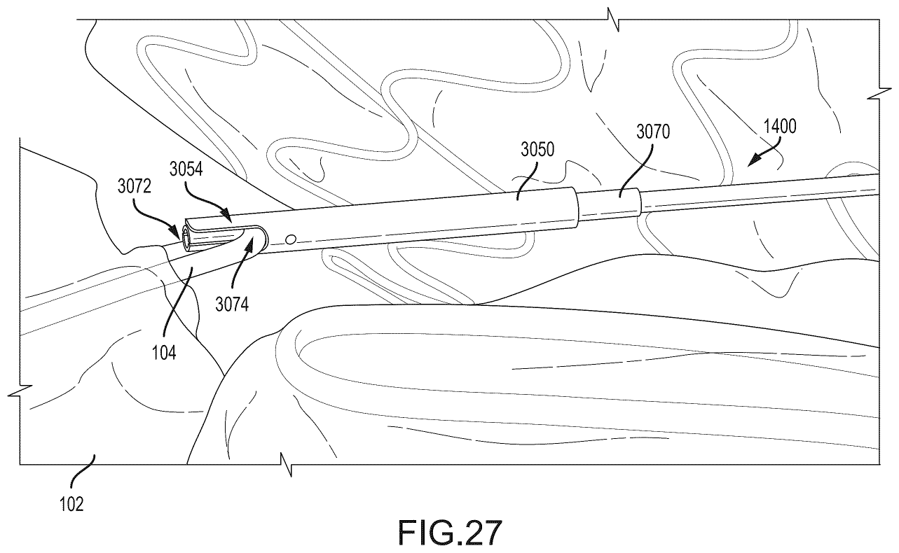

FIG. 27 shows the device of FIGS. 26A-26B in use. The graft 102 and the frame 104 may be visible, along with the guide member retainer assembly 2900 engaging with the frame 104.

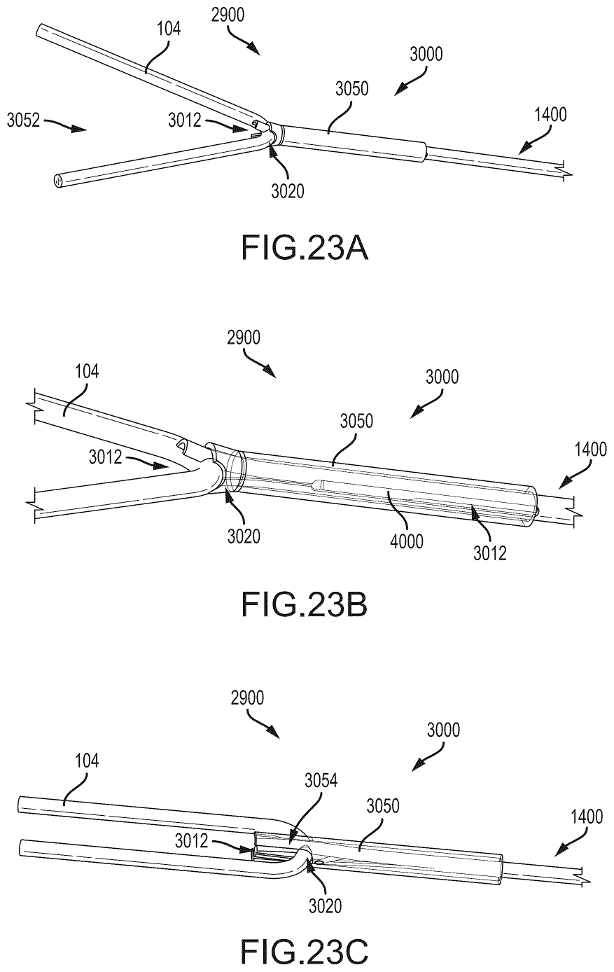

The method of using the guide member retainer assembly 2900 of FIGS. 23A-27 may begin with positioning the guide members 1400 through the side branch portals 110 of the main body 100 in a pre-cannulated configuration. The proximal hypotube 3050 may be positioned to cover and support the second proximal hypotube 3070 or the guide member 1400, depending on the specific configuration, to prevent premature release of the frame 104.

During assembly, the release wire 4000 may be positioned within the guide member 1400 and coupled to the interior surface of the proximal hypotube 3050, either directly or through the pin 4006. The frame 104 may be positioned through the retainer 3074 or frame retainer groove 3020 and covered by the proximal hypotube 3050 to secure the connection during delivery.

The delivery system 1000 may then be advanced to the target site with the guide members 1400 pre-cannulating the side branch portals 110. Once the main body 100 is positioned at the target site, the guide members 1400 may serve as rail wires to facilitate cannulation of the side branch vessels.

When cannulation of the side branch vasculature is achieved and the guide members 1400 are no longer needed, the release wire 4000 may be withdrawn from the guide member 1400. This withdrawal may cause the proximal hypotube 3050 to translate away from the frame 104, either through direct coupling or through the pin 4006 mechanism. As the proximal hypotube 3050 moves away, it may expose the side wall opening 3012 or allow the proximal end of the second proximal hypotube 3070 to splay open.

Once the constraining force of the proximal hypotube 3050 is removed, the frame 104 may be freed from the guide member retainer 3000, allowing the guide member 1400 to be withdrawn from the delivery system 1000 and removed from the patient. The translating hypotube configuration may provide secure retention during delivery while allowing for controlled release through the withdrawal motion of the release wire 4000.

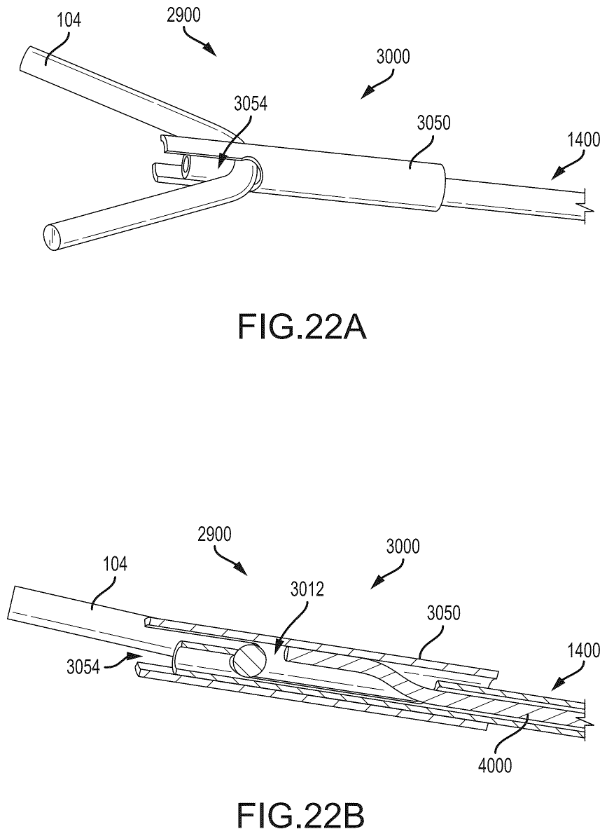

FIGS. 22A-22B and FIGS. 23A-23C illustrate orthogonal and section views of guide member retainer assemblies 2900. Referring specifically to FIGS. 22A-22B, the guide member retainer assembly 2900 may include a guide member 1400 and a guide member retainer 3000. The guide member retainer 3000 may comprise a proximal hypotube 3050 having a slot 3054 and positioned longitudinally moveable about a portion of the guide member 1400.

In some cases, the guide member 1400 may include a side wall opening 3012. The release wire 4000 may extend through the guide member 1400 and out the side wall opening 3012. The release wire 4000 may be coupled to an interior surface of the proximal hypotube 3050.

The frame 104 may be positioned through the side wall opening 3012 of the guide member 1400 and the slot 3054 of the proximal hypotube 3050. In some cases, the frame 104 may be covered by the proximal hypotube 3050 to restrict removal of the frame 104 from the side wall opening 3012 of the guide member retainer 3000.

As the release wire 4000 is withdrawn, the proximal hypotube 3050 translates away from the frame 104. This translation may at least partially reveal the side wall opening 3012, thus allowing the frame 104 to be freed from the guide member retainer 3000.

FIGS. 23A-23C illustrate a variation of the guide member retainer assembly 2900. In this variation, the guide member 1400 may include a frame retainer groove 3020. The side wall opening 3012 may extend to the proximal end of the guide member 1400, thus allowing the proximal end of the guide member 1400 to splay open to release the frame 104.

The frame 104 may be positioned through the frame retainer groove 3020 of the guide member 1400 and the slot 3054 of the proximal hypotube 3050. The frame 104 may be covered by the proximal hypotube 3050 to restrict removal of the frame 104 from the guide member retainer 3000.

As the release wire 4000 is withdrawn, the proximal hypotube 3050 translates away from the frame 104. This translation may allow the proximal end of the guide member 1400 to splay open, thus allowing the frame 104 to be freed from the guide member retainer 3000.

In some cases, the proximal hypotube 3050 may be laser cut to create the slot 3054. The laser cutting process may allow for precise shaping of the slot 3054 to accommodate the frame 104 and facilitate smooth translation of the proximal hypotube 3050 relative to the guide member 1400.

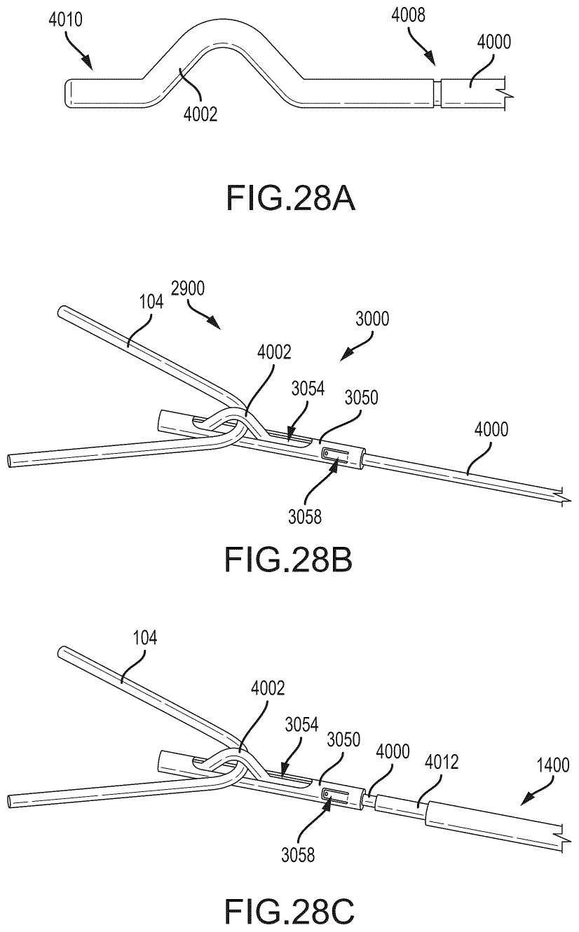

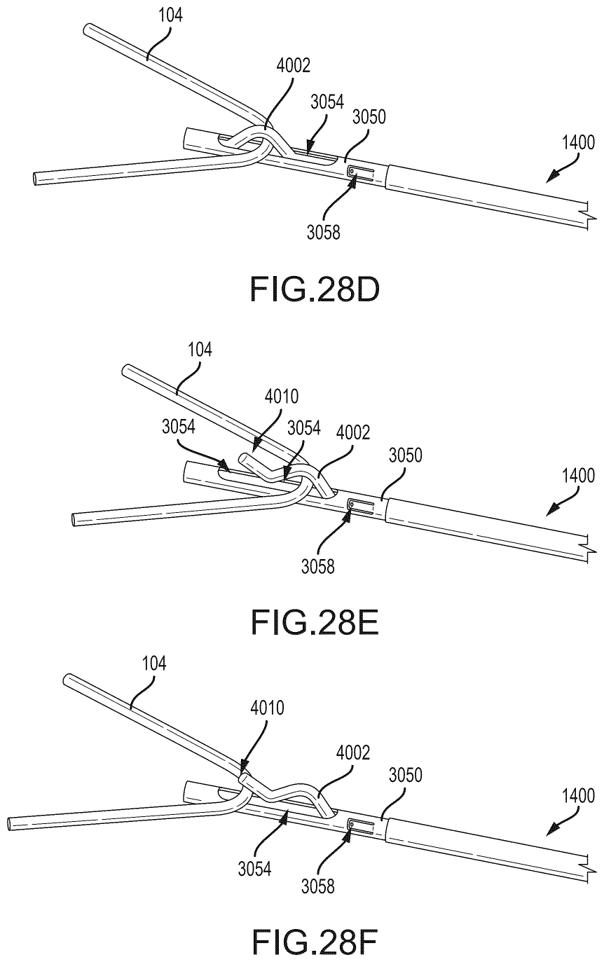

FIGS. 28A-28F illustrate various views of a guide member retainer assembly 2900 showing a release sequence. The guide member retainer assembly 2900 may include a guide member retainer 3000 coupled to the frame 104. The guide member retainer 3000 may include a proximal hypotube 3050 that includes a slot 3054 and a retention tab 3058. The release wire 4000 extends through the proximal hypotube 3050 and may include a curved portion 4002 that protrudes from the slot 3054, a groove 4008 that is engaged by the retention tab 3058, and a proximal end 4010 which, when released from the proximal hypotube 3050, may free the frame 104 from the guide member retainer 3000 so the guide member 1400 can be withdrawn.

In FIG. 28A, the release wire 4000 is shown with the curved portion 4002 and the groove 4008 in detail. The curved portion 4002 is configured extend through the slot 3054 to engage with the frame 104, while the groove 4008 is configured to be positioned to interact with the retention tab 3058.

FIGS. 28B-28F show a progressive disengagement sequence of the guide member retainer 3000 from the frame 104. FIG. 28B may show the assembly in the delivery configuration in which the release wire 4000 is positioned with the proximal hypotube 3050. The proximal end 4010 of the release wire 4000 is constrained by the proximal hypotube 3050, the curved portion 4002 protruding from the slot 3054 of the proximal hypotube 3050 (and coupled to the frame 104), and the groove 4008 positioned and engaged with the retention tabs 3058 of proximal hypotube 3050. FIG. 28C may illustrate the first step of the release process, which may include advancing a disengagement tube 4012 toward the proximal hypotube 3050. As the disengagement tube 4012 moves forward and engages the retention tabs 3058, the disengagement tube 4012 causes the retention tab 3058 to disengage from the groove 4008 (FIG. 28D), allowing the release wire 4000 to be withdrawn resulting in the proximal end 4010 of the release wire 4000 being freed from the proximal hypotube 3050 (FIG. 28E). With the proximal end 4010 of the release wire 4000 free, the frame 104 may be released from the guide member retainer assembly 2900 (FIG. 28F).

It is understood that any number of retention tabs 3058 may be implemented. In some embodiments, the retention tabs 3058 are laser cut from the proximal hypotube 3050 and bent inward. The bend of the retention tabs 3058 defines a ramp surface along which the disengagement tube 4012 engages. The disengagement tube 4012 translates along the ramp surface and bends or deflects the retention tabs 3058 away from the groove 4008 in order to release the release wire 4000 from its longitudinally constrained position relative to the proximal hypotube 3050.

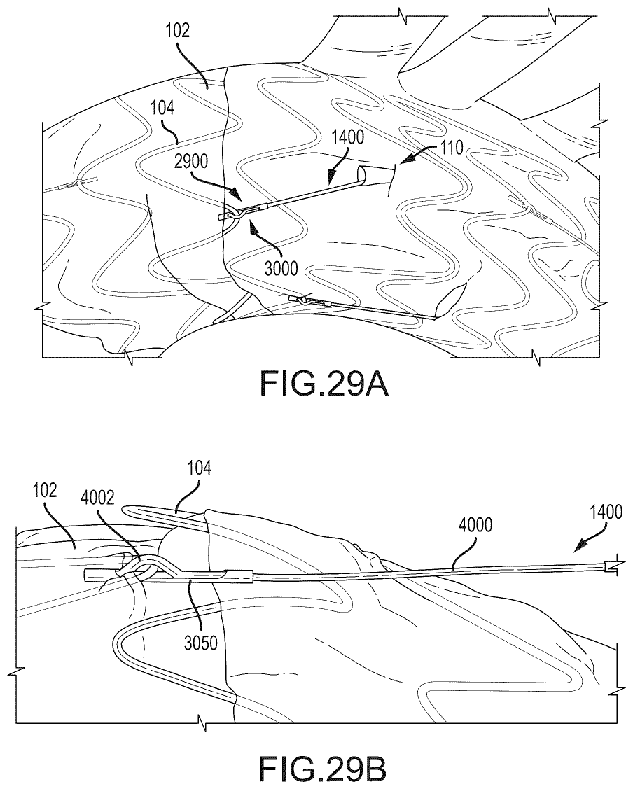

FIGS. 29A-29B are photographs of the guide member retainer assembly 2900 of FIGS. 28A-28F coupled to the frame 104. These photographs may provide a visual representation of the actual components and their arrangement in the assembled state.

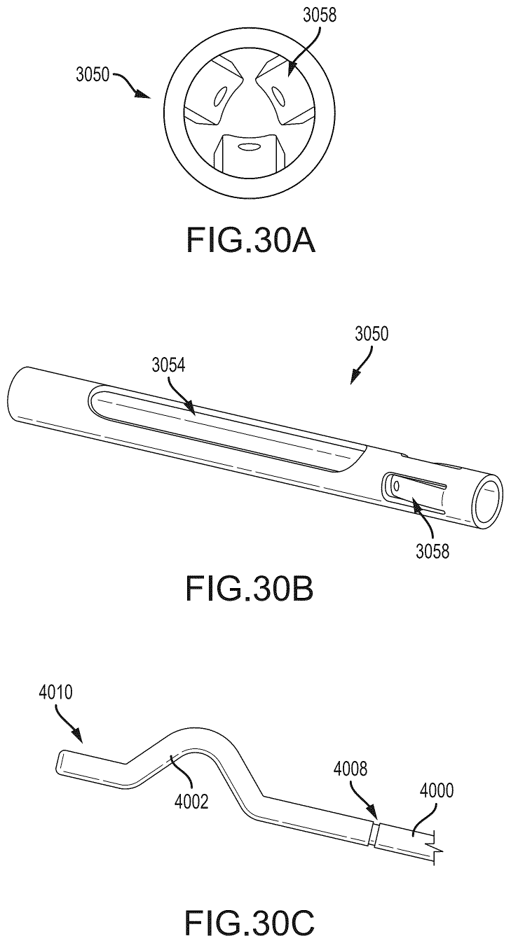

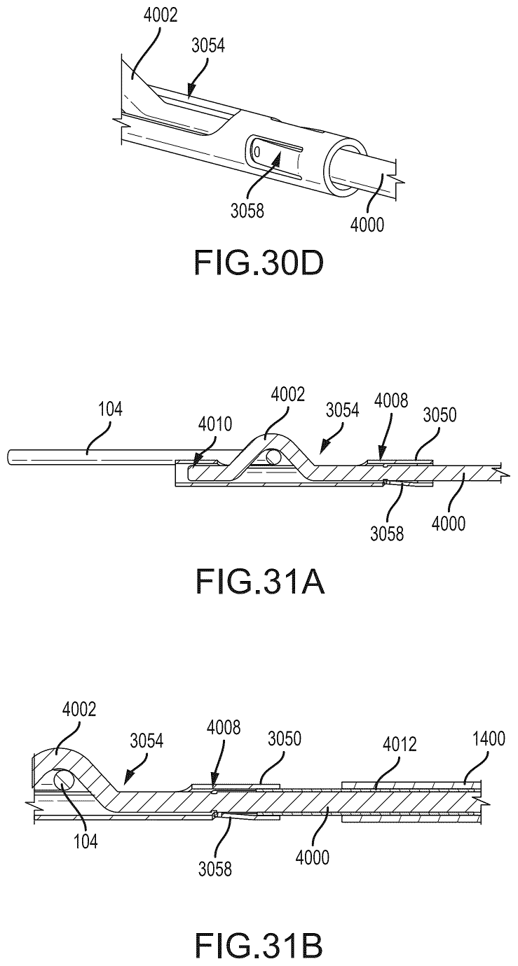

FIGS. 30A-30D illustrate several views of the guide member retainer assembly 2900 of FIGS. 28-29 in more detail. FIG. 30A shows an end view of the proximal hypotube 3050 with the retention tabs 3058 extending into the lumen of the proximal hypotube 3050. FIG. 30B depicts an orthogonal view of the proximal hypotube 3050 including the slot 3054 formed through its sidewall and the retention tabs 3058 bent inward.

FIG. 30C depicts the release wire 4000 with the curved portion 4002 for interfacing with the frame 104 and the groove 4008 configured to engage with the retention tab 3058. The release wire 4000 may extend to the proximal end 4010.

FIG. 30D depicts the interaction between the proximal hypotube 3050 and the release wire 4000. In this view, the retention tab 3058 is shown engaging with the groove 4008 of the release wire 4000, illustrating how these components interact to secure the release wire 4000 within the proximal hypotube 3050.

FIGS. 31A and 31B illustrate orthogonal and section views of the guide member retainer assembly 2900. FIG. 31A shows the proximal hypotube 3050 having the slot 3054 formed therein. The release wire 4000 extends through the proximal hypotube 3050 and includes the curved portion 4002 that protrudes through the slot 3054. The curved portion 4002 may engage with the frame 104. The retention tab 3058 extends inward from the proximal hypotube 3050 and is configured to engage with the groove 4008 formed in the release wire 4000 near the proximal end 4010.

FIG. 31B shows the guide member retainer assembly 2900 with the guide member 1400 positioned alongside the proximal hypotube 3050. The disengagement tube 4012 may be shown positioned over a portion of the release wire 4000. When the disengagement tube 4012 is advanced proximally, the disengagement tube 4012 is configured to disengage the retention tab 3058 from the groove 4008, allowing the release wire 4000 to be withdrawn proximally to release the curved portion 4002 from engagement with the frame 104.

In some cases, the retention tab 3058 and groove 4008 mechanism may provide a secure means of retaining the release wire 4000 within the proximal hypotube 3050 during delivery, while also allowing for controlled release when desired. The disengagement tube 4012 may provide a means of actuating the release mechanism without directly manipulating the retention tab 3058 or the release wire 4000, which may enhance the reliability and ease of use of the guide member retainer assembly 2900.

The method of using the guide member retainer assembly 2900 of FIGS. 28A-31B may begin with positioning the guide members 1400 through the side branch portals 110 of the main body 100 in a pre-cannulated configuration. The proximal hypotube 3050 may be positioned with the retention tabs 3058 engaging the groove 4008 of the release wire 4000 to secure the assembly to the frame 104 during delivery.

During assembly, the release wire 4000 may be positioned within the proximal hypotube 3050 with the curved portion 4002 extending through the slot 3054 to engage the frame 104. The retention tab 3058 may be positioned to engage with the groove 4008 formed in the release wire 4000, providing secure retention of the release wire 4000 within the proximal hypotube 3050 during delivery procedures.

The delivery system 1000 may then be advanced to the target site with the guide members 1400 pre-cannulating the side branch portals 110. Once the main body 100 is positioned at the target site, the guide members 1400 may serve as rail wires to facilitate cannulation of the side branch vessels.

When cannulation of the side branch vasculature is achieved and the guide members 1400 are no longer needed, the disengagement tube 4012 may be advanced proximally toward the proximal hypotube 3050. The disengagement tube 4012 may engage the retention tabs 3058 and translate along the ramp surface defined by the bend of the retention tabs 3058. This engagement may cause the retention tabs 3058 to deflect away from the groove 4008, releasing the longitudinal constraint on the release wire 4000.

Once the retention tabs 3058 are disengaged from the groove 4008, the release wire 4000 may be withdrawn proximally from the proximal hypotube 3050. This withdrawal may cause the proximal end 4010 of the release wire 4000 to be freed from the proximal hypotube 3050, allowing the curved portion 4002 to disengage from the frame 104. With the frame 104 released from the guide member retainer assembly 2900, the guide member 1400 may be withdrawn from the delivery system 1000 and removed from the patient.

The retention tab and groove mechanism may provide secure retention during delivery while allowing for controlled and reliable release through the sequential actuation of the disengagement tube 4012 and withdrawal of the release wire 4000.

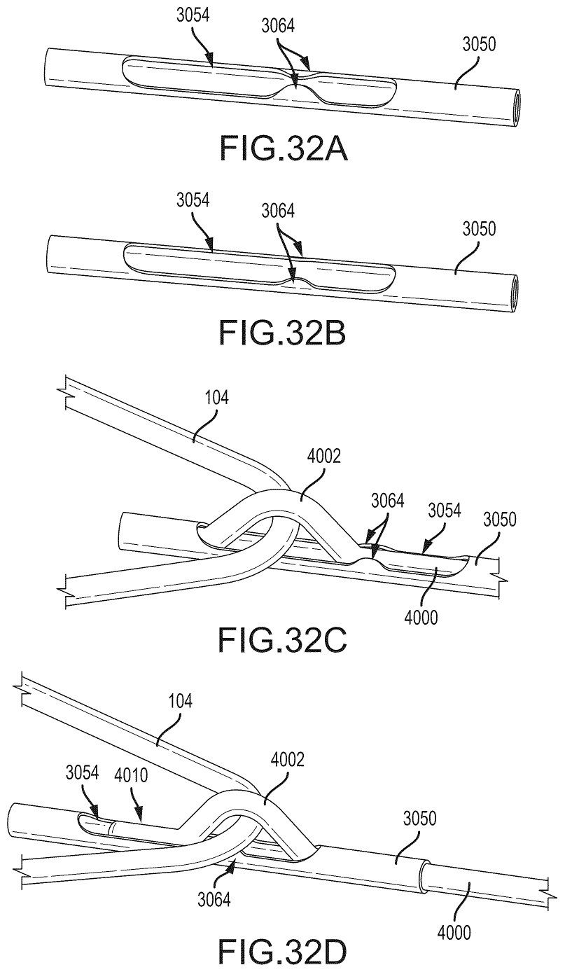

FIGS. 32A-32D illustrate various views of a guide member retainer assembly 2900.

FIG. 32A shows a side view of a proximal hypotube 3050 having a slot 3054 and retainer protrusions 3064 extending partially into the slot 3054. The retainer protrusions 3064 may be formed by cutting or otherwise removing material from the proximal hypotube 3050 to create protruding features that narrow a portion of the slot 3054 of the proximal hypotube 3050.

FIG. 32B shows another side view of the proximal hypotube 3050 with the slot 3054 and retainer protrusions 3064 where the retainer protrusions 3064 are flexed away from each other. In some cases, the retainer protrusions 3064 may be configured to flex or bend to allow passage of components through the slot 3054 of the proximal hypotube 3050 when a threshold force is applied to the retainer protrusions.

FIG. 32C shows a side view depicting the interaction between the frame 104 and the retainer assembly. The release wire 4000 extends through the proximal hypotube 3050 and includes a curved portion 4002 that extends outward from the slot 3054. The retainer protrusions 3064 may define a space narrower than a diameter of the release wire 4000, thus limiting relative movement of the release wire 4000 and the proximal hypotube 3050.

FIG. 32D shows another side view illustrating the arrangement of components including the proximal hypotube 3050 with the slot 3054 and the release wire 4000 disengaging from the frame 104. The release wire 4000 may be translated distally to force the retainer protrusions 3064 to flex to allow the release wire 4000 to translate further along the slot 3054, thus releasing the proximal end 4010 of the release wire 4000 which allows disengagement from the frame 104.

In some cases, the retainer protrusions 3064 may be configured to provide a retention force on the release wire 4000 to prevent unintended movement or release of the release wire 4000 relative to the proximal hypotube 3050. The retainer protrusions 3064 may be sized and shaped to allow passage of the release wire 4000 when sufficient force is applied, while still providing adequate retention during normal use.

The interaction between the retainer protrusions 3064 and the release wire 4000 may provide a mechanism for controlled release of the frame 104 from the proximal hypotube 3050. When the release wire 4000 is in the engaged position, the curved portion 4002 may extend through the slot 3054 to secure the frame 104. The retainer protrusions 3064 may hold the release wire 4000 in this position until intentional release is desired.

To initiate release, the release wire 4000 may be translated distally relative to the proximal hypotube 3050. This movement may cause the release wire 4000 to push against the retainer protrusions 3064, forcing them to flex or bend outward. As the retainer protrusions 3064 flex, the release wire 4000 may be able to pass through the narrowed space defined by the retainer protrusions 3064.

Once the release wire 4000 moves past the retainer protrusions 3064, the proximal end 4010 may be withdrawn from the slot 3054, allowing the frame 104 to disengage from the proximal hypotube 3050. This controlled release mechanism may allow for precise timing of frame release during the deployment procedure.

In some cases, the retainer protrusions 3064 may be formed from the same material as the proximal hypotube 3050, providing a consistent and integrated retention mechanism. The shape and size of the retainer protrusions 3064 may be optimized to provide the desired retention force while still allowing for controlled release when needed.

The guide member retainer assembly 2900 with the retainer protrusion release mechanism may provide a secure means of retaining the release wire 4000 and the frame 104 during delivery, while also allowing for controlled and intentional release when desired. This mechanism may enhance the reliability and precision of the frame release process during deployment of the implantable device 10.

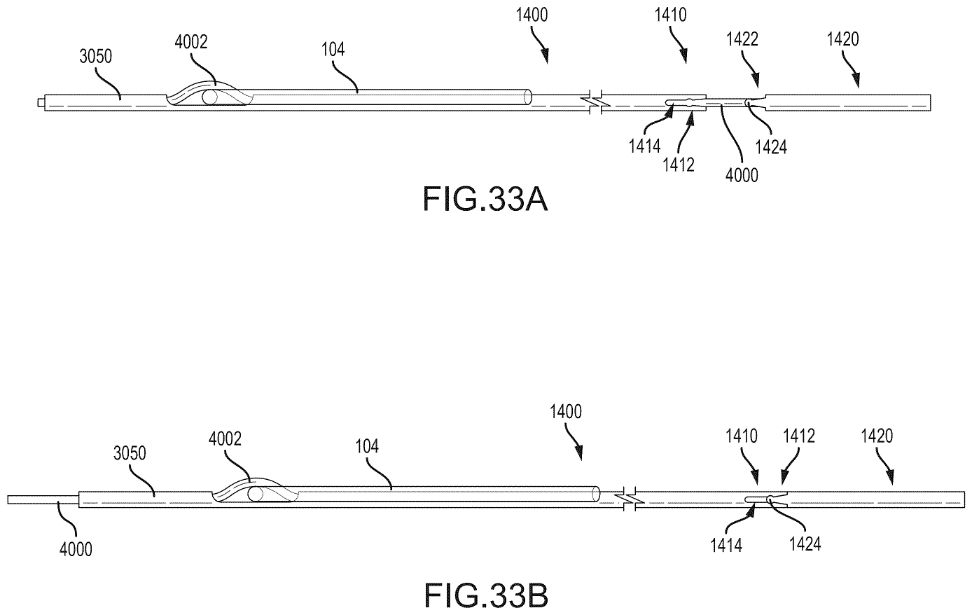

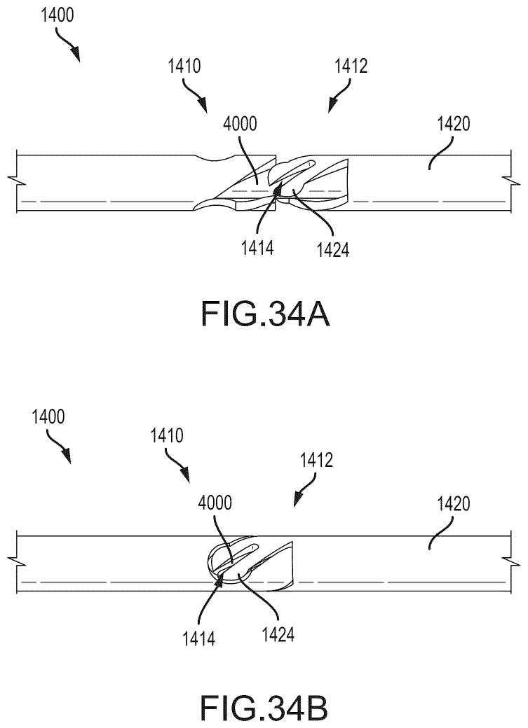

FIGS. 33A-33B and FIGS. 34A-34B illustrate side views of guide member retainer assemblies 2900. The guide member retainer assembly 2900 may include a removal member 1420 for actuating the release wire 4000. The removal member 1420 provides an interface for the user to actuate the release wire 4000.

In some cases, the guide member 1400 may include a first coupling portion 1410. The first coupling portion 1410 includes a receiver 1412. The receiver 1412 may incorporate an expansion slot 1414. The expansion slot 1414 may allow for controlled flexibility of the first coupling portion 1410.

The removal member 1420 is coupled to a distal end of the release wire 4000. The removal member 1420 includes a second coupling portion 1422. The second coupling portion 1422 is configured to interface with the first coupling portion 1410 of the guide member 1400.

In some cases, the second coupling portion 1422 of the removal member 1420 may include a retention tab 1424. The retention tab 1424 is configured to engage with the receiver 1412. The expansion slot 1414 may facilitate engagement and disengagement of the second coupling portion 1422 with the first coupling portion 1410 as a user linearly translates the removal member 1420.

FIGS. 34A-34B illustrate the guide member retainer assembly 2900 in both engaged and disengaged configurations. FIG. 34B shows the components in an assembled state where the retention tab 1424 of the removal member 1420 is engaged with the receiver 1412 of the first coupling portion 1410. In this configuration, the expansion slot 1414 may accommodate the retention tab 1424 while maintaining a secure connection between the removal member 1420 and the guide member 1400.

The release wire 4000 extends through the assembly to maintain the coupling between these components.

FIG. 34A depicts the components in a separated configuration, demonstrating how the retention tab 1424 disengages from the receiver 1412 when the release wire 4000 is withdrawn. The expansion slot 1414 may allow the first coupling portion 1410 to flex sufficiently to release the retention tab 1424 from the receiver 1412. This separation may occur when the user applies a withdrawal force to the removal member 1420, causing the release wire 4000 to be actuated and the coupling mechanism to disengage.

These arrangements may allow for controlled movement and release of the guide member 1400 while limiting accidental or unintended actuation of the release wire 4000. In some cases, the components may be reversed, with the first coupling portion 1410 on the removal member 1420 and the second coupling portion 1422 on the guide member 1400.

The first coupling portion 1410 and second coupling portion 1422 may be configured with complementary angled surfaces that require a specific rotational movement for proper engagement and disengagement. In some embodiments, the retention tab 1424 may be positioned at an angle relative to the longitudinal axis of the removal member 1420, while the receiver 1412 may include a corresponding angled channel or groove. This angled relationship may prevent accidental disengagement during linear forces applied along the axis of the guide member 1400.

The angled configuration may require the user to apply both rotational and linear forces to disengage the coupling portions. In some cases, the retention tab 1424 may be oriented at an angle between about 15 degrees and about 75 degrees relative to the longitudinal axis of the removal member 1420. The receiver 1412 may include a helical or spiral pathway that guides the retention tab 1424 during engagement and disengagement.

During disengagement, the removal member 1420 may be rotated in a predetermined direction while simultaneously being withdrawn distally. The angled surfaces of the first coupling portion 1410 and second coupling portion 1422 require the proper rotational and linear forces to be applied to achieve withdrawal. This multi-directional actuation requirement may reduce the likelihood of unintentional release during normal handling or deployment procedures.

These arrangements may allow for controlled movement and release of the guide member 1400 while limiting accidental or unintended actuation of the release wire 4000. In some cases, the components may be reversed, with the first coupling portion 1410 on the removal member 1420 and the second coupling portion 1422 on the guide member 1400.

The guide member retainer assembly 2900 may include features to differentiate between multiple guide members 1400. In some cases, the guide members 1400 may be differentiated by color. The color differentiation may be achieved through anodization of the guide members 1400. The anodization may be applied to the entire guide member 1400 or to a local portion of the guide member 1400.

In some cases, the guide members 1400 may be differentiated by heat treatment. The heat treatment may create an oxide layer on the guide members 1400. The oxide layer may be applied to the entire guide member 1400 or may be locally applied using a laser.

The guide members 1400 may be differentiated by a polymer coating in some cases. The polymer coating may be applied as a powder coating. The powder coating may be applied to ground grooves on the guide members 1400 or to the entire guide member 1400.

In some cases, the guide members 1400 may be differentiated by laser marking. The laser marking may be applied to one end or both ends of the guide members 1400. The guide members 1400 may also be differentiated by length in some cases, with three different lengths provided for the guide members 1400.

The guide member retainer assembly 2900 may include removable labels to further differentiate the guide members 1400. The removable labels may be applied to the guide members 1400 and removed after the guide members 1400 are positioned in the delivery system 1000.

The guide members 1400 and associated components may be constructed from various materials to achieve the desired mechanical properties and biocompatibility. In some cases, the guide members 1400 may comprise wires formed from metallic materials such as nitinol, stainless steel, or other suitable metals. The guide members 1400 may alternatively comprise suture materials, including both absorbable and non-absorbable sutures. In some aspects, polymeric materials may be used to form the guide members 1400, including but not limited to polyfluoromers, polyethylene, polypropylene, or other biocompatible polymers.

The release wire 4000 may similarly be constructed from various materials depending on the specific application requirements. In some cases, the release wire 4000 may comprise nitinol wire to provide shape memory characteristics. Alternatively, the release wire 4000 may be formed from stainless steel wire to provide strength and durability. Polymeric materials may also be used for the release wire 4000 in applications where flexibility or specific surface properties are desired.

The proximal hypotube 3050 and second proximal hypotube 3070 may be fabricated from metallic materials such as stainless steel or nitinol to provide the necessary structural support and flexibility. In some aspects, these components may be formed from polymeric materials where weight reduction or specific mechanical properties are advantageous. The term “hypotube” as used herein is not intended to be limiting to any particular form or construction method, and may encompass various tubular structures including drawn tubes, extruded tubes, or other hollow cylindrical components regardless of their manufacturing process or specific dimensional characteristics.

The guide member retainer 3000 and associated components may be constructed from materials that provide the appropriate balance of strength, flexibility, and biocompatibility. In some cases, metallic materials such as nitinol or stainless steel may be used to provide durability and corrosion resistance. Polymeric materials may be employed where specific surface properties or reduced weight are desired.

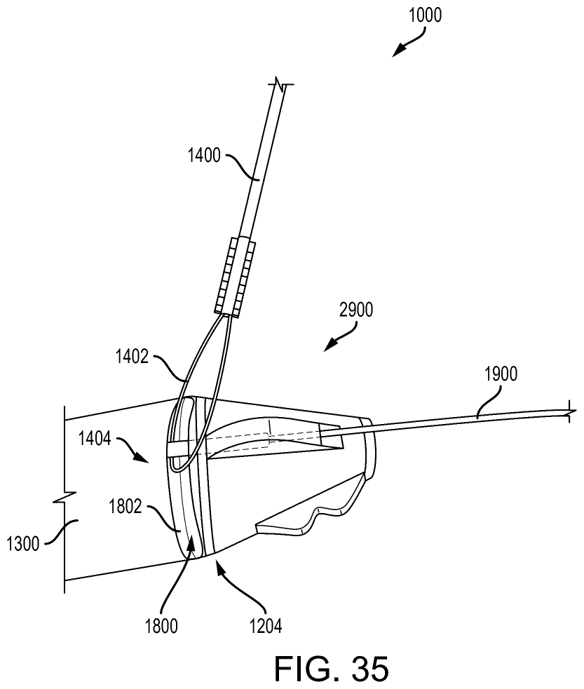

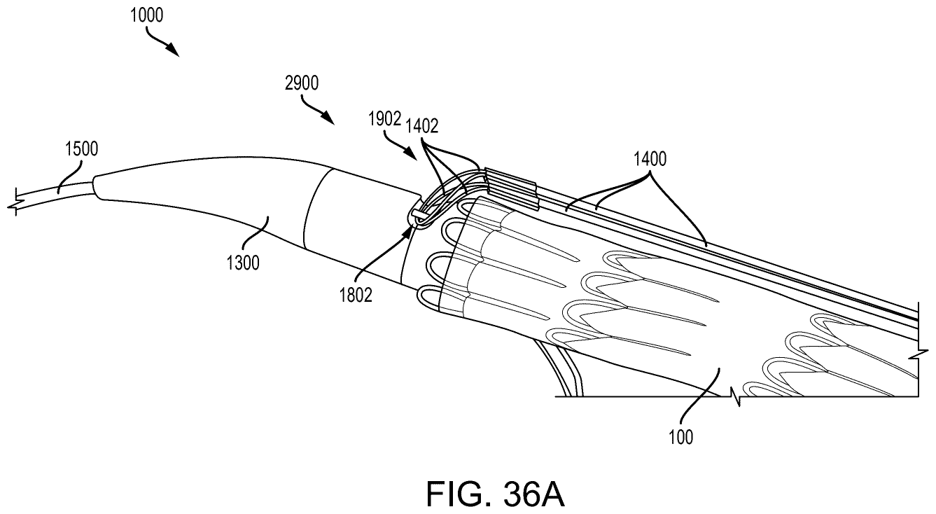

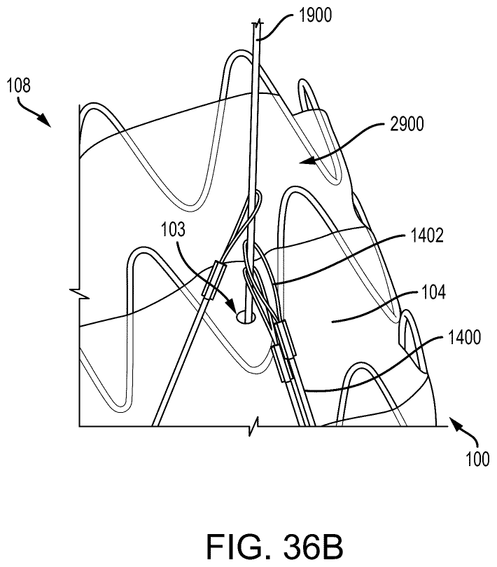

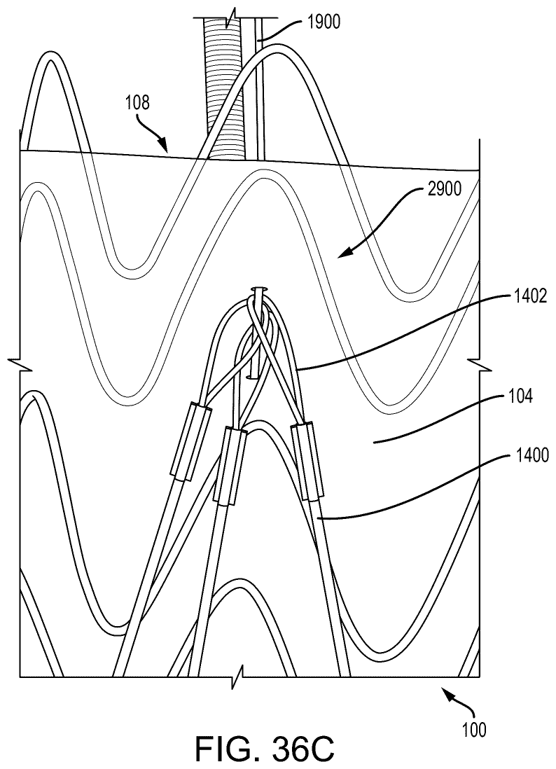

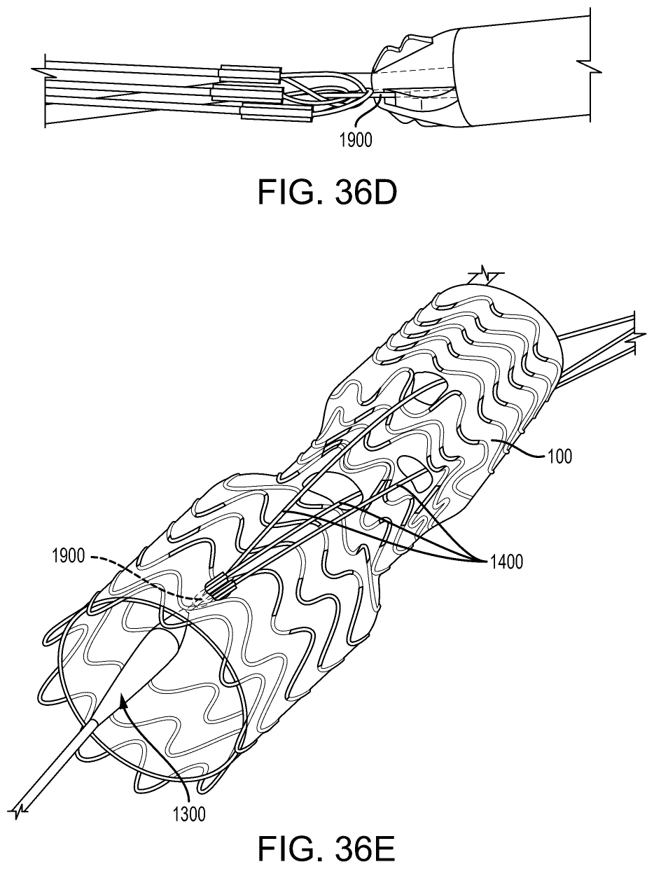

Turning to a discussion of FIGS. 35-36E, additional embodiments of a releasable guide member system are provided. Referring specifically to FIGS. 35 and 36A, the delivery system 1000 according to principles of the present disclosure can include a guide member retainer assembly 2900 that releases one or more guide members 1400, which frees the guide members 1400 to be released from the main body 100 (e.g., through the side branch portal 110 or opening). In some embodiments, the guide member retainer assembly 2900 defines a window 1802 through which guide members 1400 are configured to engage the lockwire 1900 (e.g., the window 1802 may be defined in the cap 1300). The window 1802 provides access to a portion (e.g., a longitudinal length) of the lockwire 1900 while providing stops (e.g., side walls of the cap 1300) to limit disengagement of the guide members 1400 from the lockwire 1900 while the lockwire 1900 is engaged with the cap 1300.

In some embodiments, the window 1802 is defined at a proximal end of the delivery system 1000 (e.g., at the cap 1300 or the elongate member 1200 (not shown)). The lockwire 1900 is partially exposed within the window such that the guide members 1400 can engage and be retained by the lockwire 1900. For example, the guide members 1400 may include engagement portions 1402 that are configured to be coupled to and retained by the lockwire 1900. In some embodiments, the engagement portions 1402 define loops 1404 for each guide member 1400 which are configured to surround the lockwire 1900. The engagement portion 1402 may be integral with or coupled to the guide members 1400 (e.g., a braided wire defining a loop and crimped to the guide member 1400). The guide members 1400 (e.g., the engagement portions 1402) extend into the window 1802 (see FIGS. 35 and 36A) and the lockwire 1900 is positioned through the loops 1404. In particular, FIG. 35 shows an instance (e.g., as an implementation or as a snapshot during assembly) of the delivery system 1000 where there is only one lockwire 1900 and one guide member 1400 engaged therewith. FIG. 36 shows a similar instance of the delivery system 1000 except that there is a lockwire 1900 and a plurality of guide members 1400 engaged therewith. In particular, three guide members 1400 are shown. These are just some examples of the many examples contemplated herein as there may be numerous unique combinations of windows 1802, side branch portals 110, lockwires 1900, and guide members 1400. For instance, this disclosure contemplates examples where a number of lockwires 1900 corresponds to a number of guide members 1400.

Referring to FIGS. 36B and 36C, in some embodiments, the guide member retainer assembly 2900 includes various components for selectively releasing guide members 1400. For example, the lockwire 1900 can extend through at least a portion of the graft material 104 of the main body 100. The lockwire 1900 can be threaded through an aperture 103 (see FIG. 36B) in the graft material 104 or may be sewn through a portion of the graft material 104 (see FIG. 36C). The lockwire 1900 may be exposed such that the engagement portions 1402 of the guide members 1400 are engaged with the lockwire 1900. For example, referring to FIG. 36B, the lockwire 1900 passes from the main body lumen 108, through the aperture 103 positioned between ends of the main body 100, and extends to an exterior of the main body 100. The engagement portions 1402 of the guide members 1400 engage the lockwire 1900 on the exterior side of the main body 100. The end of the lockwire 1900 may be constrained by another component of the delivery system 1000, such as the cap 1300 (e.g., tip and/or olive) to reduce potential trauma that could be caused by a free end of the lockwire 1900 (see FIGS. 36D and 36E). Referring to FIG. 36C, the lockwire 1900 passes from the main body lumen 108, extends through the graft material 104 (e.g., at a first longitudinal position), traverses along a portion of the longitudinal length of the main body 100, and extends back through the graft material 104 (e.g., at a second longitudinal position) into the main body lumen 108. The portion of the lockwire 1900 exposed on the exterior of the graft material 104 are engaged by the engagement portions 1402 of the guide members 1400 are engaged with the lockwire 1900. The end of the lockwire 1900 may be constrained by another component of the delivery system 1000, such as the cap 1300 to reduce potential trauma that could be caused by a free end of the lockwire 1900. In each of these embodiments, the lockwire 1900 is engaged (e.g., actuated away from the cap 1300) in order to free the engagement portions 1402 of the guide members 1400.

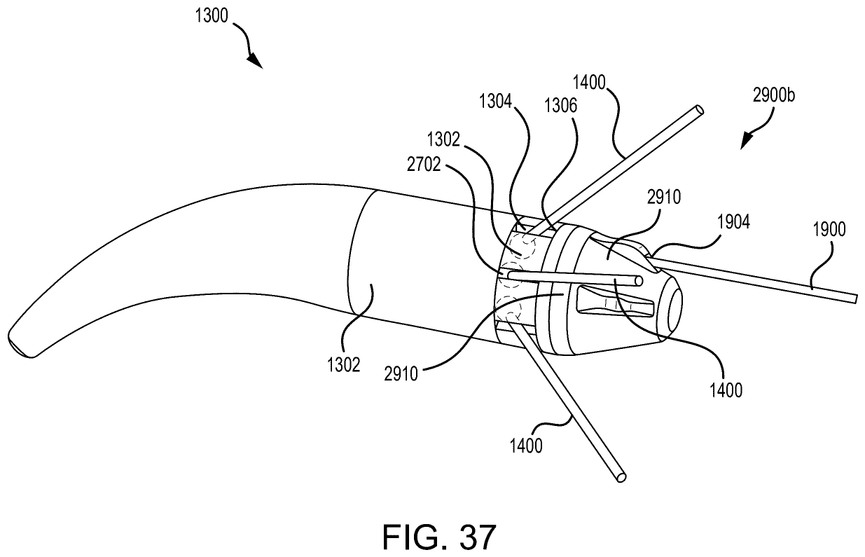

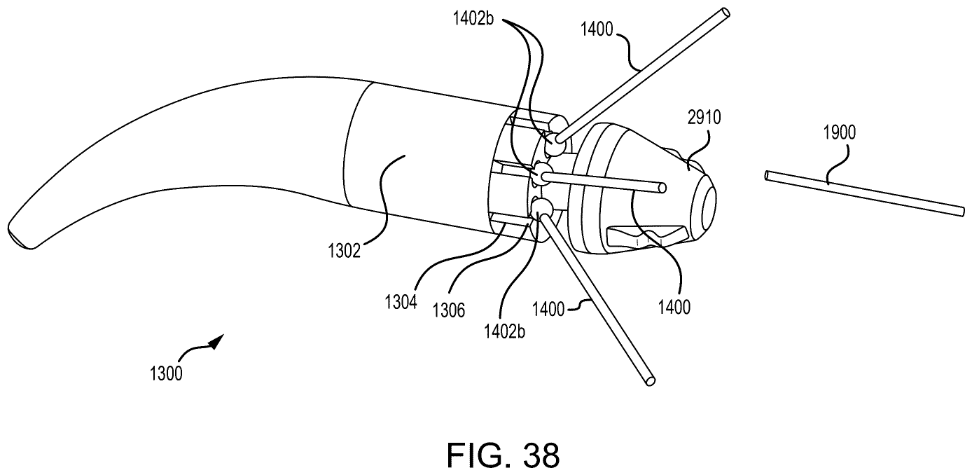

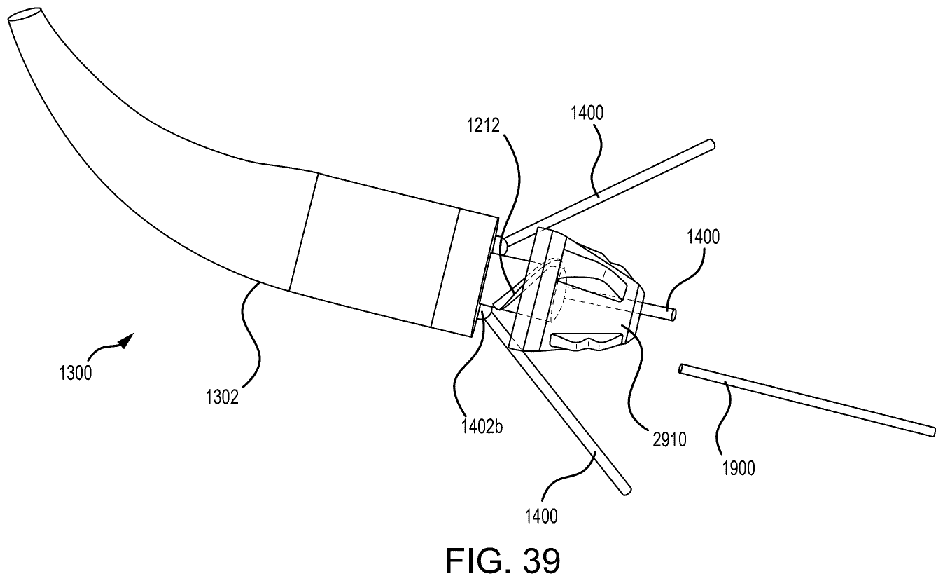

Referring now to FIGS. 37-39, in some embodiments, the delivery system 1000 includes a guide member retainer assembly 2900b positioned at the cap 1300. For example, the cap 1300 includes a cap body and movable collar 2910 positioned adjacent the end cap body 1302. The movable collar 2910 is movable relative to other components of the delivery system 1000 (e.g., from a coupling portion or threaded portion of the delivery system 1000 such as the cap 1300 or elongate member 1200 (not shown)). As illustrated, the movable collar 2910 defines a portion of the delivery system 1000 (e.g., a portion of the cap 1300). In particular, FIG. 37 shows the guide member retainer assembly 2900b where the movable collar 2910 is secured to the end cap body 1302. FIG. 38 shows the guide member retainer assembly 2900b of FIG. 37 where the movable collar 2910 is partially removed from the end cap body 1302. And FIG. 39 shows the guide member retainer assembly 2900 of FIG. 37 where the movable collar 2910 is partially removed from the cap 1300 and made transparent for illustration purposes to reveal the threaded engagement between the movable collar 2910 and the cap 1300.

The guide members 1400 can be captively held by the guide member retainer assembly 2900b and are releasable via manipulation of the delivery system 1000. For instance, the delivery system 1000 (e.g., the cap 1300) can have the orifice formed as a slot 1304 that includes a free end 1306 through which the guide member 1400 is received to be secured at an engagement portion 1402b of the guide member 1400. The engagement portion 1402b can be an embossment that is sized to inhibit the guide member 1400 from being released through the orifice. The slot 1304 is formed through at least a portion of a thickness of the end cap body 1302.

A lockwire 1900 of the guide member retainer assembly 2900 is received through a lockwire orifice 1904 formed in the movable collar 2910 and at least a portion of the cap 1300. When the lockwire 1900 is positioned through the lockwire orifice, the movable collar 2910 is restrained from movement relative to the cap 1300. The movable collar 2910 is capable of moving relative to the elongate member 1200 and the cap 1300 when the lockwire 1900 is withdrawn through the lockwire orifice 1904. In some embodiments, this is because the movable collar 2910 is in threaded engagement with the cap 1300 at a thread 1212. Because the lockwire limits rotational movement of the movable collar 2910 relative to the cap 1300, the movable collar 2910 is not able to withdraw from the threaded engagement with the cap 1300 via the thread 1212 until the lockwire 1900 is removed and the movable collar 2910 is free to rotate relative to the cap 1300. In this regard, withdrawal of the lockwire 1900 allows the movable collar 2910 to unthread from the thread 1212. In some embodiments, the thread 1212 is provided at a pitch that facilitates longitudinal force to be translated into rotational movement of the movable collar 2910. For example, the movable collar 2910 is unthreaded from the end cap body 1302 by tensioning the guide member 1400. The thread 1212 and movable collar 2910 may be provided in materials or include a lubricious coating in order to facilitate translation of the longitudinal force into rotational movement of the movable collar 2910 relative to the cap 1300 along the thread 1212. Although the thread is shown as defined on the cap 1300, it is understood that the thread may be defined on the elongate member 1200 or any other appropriate structure.

In a practical example, the delivery system 1000 includes a laserball helical collar release function. For example, the engagement portion 1402b may define laserball attachment points formed on the end of the guide member 1400. These laserballs can then be located on an internal feature on the cap 1300 (e.g., orifice formed as the slot 1304 that includes the free end 1306) so that after the lockwire 1900 is removed, the laserballs have enough space to come out or the from the slot 1304 via the free end 1306. Here, guide members 1400 are retained using laserball ends captive in slot features of the tip/olive. A helically threaded movable collar pinned with a lockwire 1900 keeps the movable collar 2910 from spinning and moving axially. A thread pitch is selected to be large enough that pulling on the rail wires would rotate the movable collar once the lockwire 1900 is removed.

Implementations may include one or more of the following features. When assembled, a portion of the guide member 1400 at the movable collar 2910 can extend away at an angle to the central axis of the elongate member body 1201. Radially, the guide member 1400 and the lockwire 1900 can be bundled proximate to each other at a radial segment of the elongate member body 1201. When there is a plurality of guide members 1400, each of the guide members 1400 can be bundled proximate to each other and to the lockwire 1900 at a radial segment of the elongate member body 1201. The radial segment can be less than 180 degrees.

A deployment method may include advancing a main guidewire 1500 to a target site. The method may include advancing an elongate member 1200 (e.g., a catheter) of a delivery system 1000 for a main body 100 along the main guidewire 1500 toward the main lumen of the target site, the main body 100 defining at least one side branch portal 110. A corresponding guide member 1400 is cannulating each side branch portal 110 and is retained by the cap 1300 as described above. The method may include performing a multistage endoprosthesis deployment process in which branch bodies 200 are positioned in the corresponding side branch vasculature as previously described using the guide members 1400. When the guide members 1400 are no longer needed for positioned the branch bodies 200, the lockwire 1900 of a guide member retainer assembly 2900 is actuated which facilitates release of at least one guide member 1400 and allows the at least one guide member 1400 to be retracted along the elongate member 1200. The multistage endoprosthesis deployment process can include tensioning the at least one guide member 1400 to thereby cause the movable collar 2910 of the catheter to decouple to release the at least one guide.

The selection of materials for each component may be optimized based on factors such as the intended deployment environment, required mechanical properties, manufacturing considerations, and biocompatibility requirements. The various materials may be used individually or in combination to achieve the desired performance characteristics for each specific application.

Delivery systems and methods disclosed herein are particularly suited for endoluminal delivery of branchable expandable implants for treating branched vasculature. Expandable implants can include, for example, stents, grafts, and stent grafts. Further, expandable implants can include one or more stent components with one or more graft members disposed over and/or under the stent, which can dilate from a delivery configuration, through a range of larger intermediary configurations, and toward a deployed configuration engaged with vessel walls at a treatment site. However, and as discussed below, any suitable combination and configuration of stent component(s) and graft member(s) is within the scope of the present disclosure. For example, stent components can have various configurations such as, for example, rings, cut tubes, wound wires (or ribbons) or flat patterned sheets rolled into a tubular form. Stent components can be formed from metallic, polymeric or natural materials and can comprise conventional medical grade materials such as nylon, polyacrylamide, polycarbonate, polyethylene, polyformaldehyde, polymethylmethacrylate, polypropylene, polytetrafluoroethylene, polytrifluorochlorethylene, polyvinylchloride, polyurethane, elastomeric organosilicon polymers; metals such as stainless steels, cobalt-chromium alloys and nitinol and biologically derived materials such as bovine arteries/veins, pericardium and collagen. Stent components can also comprise bioresorbable materials such as poly(amino acids), poly(anhydrides), poly(caprolactones), poly(lactic/glycolic acid) polymers, poly(hydroxybutyrates) and poly(orthoesters).

Moreover, potential materials for graft members include, for example, expanded polytetrafluoroethylene (ePTFE), polyester, polyurethane, fluoropolymers, such as perfluoroelastomers and the like, polytetrafluoroethylene, silicones, urethanes, ultra-high molecular weight polyethylene, aramid fibers, and combinations thereof.

Other embodiments for a graft member material can include high strength polymer fibers such as ultra-high molecular weight polyethylene fibers (e.g., Spectra®, Dyneema Purity®, etc.) or aramid fibers (e.g., Technora®, etc.). The graft member may include a bioactive agent. In one embodiment, an ePTFE graft includes a carbon component along a blood contacting surface thereof. Any graft member which can be delivered by a catheter is in accordance with the present disclosure.

In addition, nitinol (NiTi) may be used as the material of the frame or stent (and any of the frames discussed herein), but other materials such as, but not limited to, stainless steel, L605 steel, polymers, MP35N steel, polymeric materials, Pyhnox, Elgiloy, or any other appropriate biocompatible material, and combinations thereof, can be used as the material of the frame. The super-elastic properties and softness of NiTi may enhance the conformability of the stent. In addition, NiTi can be shape-set into a desired shape. That is, NiTi can be shape-set so that the frame tends to self-expand into a desired shape when the frame is unconstrained, such as when the frame is deployed out from a delivery system. Other materials may also be used as appropriate, including but not limited to NiTiCo.

The invention of this application has been described above both generically and with regard to specific embodiments. It will be apparent to those skilled in the art that various modifications and variations can be made in the embodiments without departing from the scope of the disclosure. Thus, it is intended that the embodiments cover the modifications and variations of this invention provided they come within the scope of the appended claims and their equivalents.

Numerous characteristics and advantages of the present invention have been set forth in the preceding description, including preferred and alternate embodiments together with details of the structure and function of the invention. The disclosure is intended as illustrative only and as such is not intended to be exhaustive. It will be evident to those skilled in the art that various modifications may be made, especially in matters of structure, materials, elements, components, shape, size and arrangement of parts within the principals of the invention, to the full extent indicated by the broad, general meaning of the terms in which the appended claims are expressed. To the extent that these various modifications do not depart from the spirit and scope of the appended claims, they are intended to be encompassed therein. In addition to being directed to the embodiments described above and claimed below, the present invention is further directed to embodiments having different combinations of the features described above and claimed below.

The invention of this application has been described above both generically and with regard to specific embodiments. It will be apparent to those skilled in the art that various modifications and variations can be made in the embodiments without departing from the scope of the disclosure. Thus, it is intended that the embodiments cover the modifications and variations of this invention provided they come within the scope of the appended claims and their equivalents.

1. A delivery system for an implantable device, comprising:
an elongate member;
a main body coupled to the elongate member, the main body including a main body wall defining a main body lumen and a side branch portal through the main body wall;
a guide member extending along at least a portion of the elongate member, into the main body lumen, and through the side branch portal;
a guide member retainer assembly including:
a retainer coupled to the guide member and defining a retainer eyelet; and
a release wire extending through a portion of the main body wall and the retainer eyelet to engage the retainer eyelet such that the guide member is restricted from being retracted along the elongate member by the engagement between the release wire with the retainer eyelet.

2. The delivery system of claim 1, wherein the retainer is defined at a proximal end of the guide member.

3. The delivery system of claim 2, wherein the retainer eyelet is defined through a body of the proximal end of the guide member.

4. The delivery system of claim 1, wherein the guide member retainer assembly includes a plurality of retainers arranged in a nested configuration.

5. The delivery system of claim 4, wherein the plurality of retainers are positioned at different longitudinal positions along the release wire.

6. The delivery system of claim 5, wherein the plurality of retainers are positioned relative to each other to distribute multiple guide members in a manner that corresponds to longitudinal and angular positioning of multiple side branch portals in the main body.

7. The delivery system of claim 1, wherein the retainer has a thinner profile than a remainder of the guide member.

8. A delivery system for an implantable device, comprising:
an elongate member;
a main body positioned about the elongate member, the main body including a frame;
a guide member extending along at least a portion of the elongate member;
a guide member retainer assembly releasably coupling the guide member to the frame, the guide member retainer assembly including:
a retainer coupled to the guide member and defining a side wall opening; and
a release wire extending through the guide member and the side wall opening such that the frame is positioned between the guide member and the release wire at the side wall opening to releasably couple the guide member to the frame.

9. The delivery system of claim 8, wherein the retainer comprises a proximal hypotube coupled to a proximal end of the guide member.

10. The delivery system of claim 9, wherein the retainer further comprises a second proximal hypotube positioned within the proximal hypotube, the proximal hypotube and the second proximal hypotube configured to translate relative to each other to release the frame.

11. The delivery system of claim 9, wherein the retainer further comprises a first side wall opening and a second side wall opening positioned along a length of the proximal hypotube, wherein a frame retainer groove is formed between the first and second side wall openings.

12. The delivery system of claim 11, wherein the release wire extends through the guide member, out the first side wall opening, and back into the second side wall opening to secure the frame in the frame retainer groove.

13. The delivery system of claim 12, wherein the retainer further comprises a loop coupled to the proximal hypotube, the loop including a retainer ball, wherein the proximal hypotube includes a sidewall aperture configured to receive the retainer ball, and wherein the release wire engages the retainer ball to secure the frame until the release wire is withdrawn.

14. The delivery system of claim 9, wherein the proximal hypotube further comprises retainer protrusions extending partially into the side wall opening, the retainer protrusions configured to flex to allow passage of the release wire through the side wall opening when a threshold force is applied.

15. A delivery system for an implantable device, comprising:
an elongate member;
a main body positioned about the elongate member, the main body including a frame and defining a side branch member;
a guide member positioned through the side branch member in a pre-cannulated configuration;
a guide member retainer assembly releasably coupling the guide member to the frame, the guide member retainer assembly including:
a proximal hypotube including a slot and a retention tab; and
a release wire extending through the proximal hypotube, the release wire including a portion protruding from the slot and a groove engaged by the retention tab.

16. The delivery system of claim 15, further comprising a disengagement tube configured to disengage the retention tab from the groove when advanced proximally, allowing the release wire to be withdrawn to release the frame.

17. The delivery system of claim 16, wherein the retention tab bends inwardly to define a ramp surface.

18. The delivery system of claim 17, wherein the disengagement tube is configured to translate along the ramp surface and deflect the retention tab away from the groove to release the release wire from its longitudinally constrained position relative to the proximal hypotube.

19. The delivery system of claim 15, wherein the release wire includes a proximal end that is constrained by the proximal hypotube when the retention tab is engaged with the groove, and wherein withdrawal of the release wire results in the proximal end being freed from the proximal hypotube.

20. The delivery system of claim 15, wherein the curved portion is configured to engage with the frame when the retention tab is engaged with the groove, and wherein advancement of the disengagement tube causes progressive disengagement of the retention tab from the groove to allow withdrawal of the release wire and release of the frame from the guide member retainer assembly.
